(19) United States
(12) Patent Application Publication
(10) Pub. No.: US 2026/0069529 A1
(43) Pub. Date: Mar. 12, 2026

(54) TOPICAL COMPOSITIONS OF MICROCOCCUS LUTEUS Q24 AND USE THEREOF

(71) Applicant: BLIS TECHNOLOGIES LIMITED, Dunedin (NZ)

(72) Inventors: Rohit JAIN, Dunedin (NZ); John David Francis HALE, Dunedin (NZ); Sonali Sudhir SALI, Dunedin (NZ); Stephanie Anne MATTISON, Dunedin (NZ)

(21) Appl. No.: 19/108,267

(22) PCT Filed: Sep. 1, 2023
(86) PCT No.: PCT/IB2023/058645

§ 371 Date: Mar. 3, 2025

(30) Foreign Application Priority Data
Sep. 2, 2022 (AU) 2022902530

(51) Int. Cl. A61K 8/99; A61K 8/31; A61K 8/67; A61K 8/92; A61Q 19/00; A61Q 19/08

(52) U.S. Cl. CPC A61K 8/99; A61K 8/31; A61K 8/678; A61K 8/922; A61Q 19/00; A61Q 19/007; A61Q 19/08; A61K 2800/48

(57) ABSTRACT

The present invention relates to topical compositions comprising Micrococcus luteus Q24 and a viscosity modifier, the composition having a viscosity of from about 2,500,000 to about 8,000,000 cP at 25° C., and the use of these for improving the appearance of skin or at least one sign of aging, or reducing skin dryness.

This application claims priority to Australian Provisional Application No. 2022902530, filed 2 Sep. 2022, the contents of which are herein incorporated by reference.

### FIELD OF THE INVENTION

The present invention relates to Micrococcus luteus compositions and methods of using the compositions to improve the appearance of skin, or at least one sign of skin aging. The invention also relates to methods of making the compositions.

### BACKGROUND TO THE INVENTION

Skin-care products containing probiotic microorganisms are becoming increasingly well-known. The microorganisms or related products used in skin-care products to date are generally Bifidobacterium spp., Lactobacillus spp. (now known as, Limosilactobacillus spp., Lacticaseibacillus spp., Lactiplantibacillus spp., and Ligilactobacillus spp.), Lactococcus spp., and Streptococcus spp., or filtrates or lysates etc. created from the bacteria. However, the role they play in interacting with the skin microbiome is not well understood.

WO2006104403 (Blis Technologies Limited) describes Micrococcus luteus (M. luteus) compositions and their therapeutic use for controlling skin diseases or disorders. Probiotic strain Q24 on deposit at Deutsche Sammlung von Mikroorganismen und Zellkulturen GmbH, Braunschweig, Germany, under accession number DSM 17172 is also provided. This document is incorporated herein by reference in its entirety. WO2006104403 discloses a saline suspension, a grapeseed oil suspension and a hard deodorant stick.

ANZCTR, A Probiotic for Eczema Treatment (Registration number: ACTRN12616000022460) describes a clinical trial to test the use of a lysate of Micrococcus luteus Q24 for the treatment of eczema. No results are provided.

The applicants have unexpectedly identified a new role for Micrococcus luteus in cosmetic applications. Such cosmetic applications include improving skin appearance, or at least one sign of skin aging. Based on this unexpected finding, there is a need to develop new cosmetic treatment methods and compositions suitable for cosmetic use.

Working with probiotic organisms is challenging. Probiotics are sensitive and often respond unpredictably to a range of freeze-drying processes, agents used in such processes, processing conditions (such as pressure or elevated temperatures), and formulation agents, amongst others.

In the harsh freeze-drying processes, microorganisms are generally protected using a range of lyoprotectants and cryoprotectants. The applicants have also surprisingly found that Micrococcus luteus can be formulated in the absence of a lyoprotectant or cryoprotectant without significant loss in viability of the probiotic microorganism.

Preparing compositions comprising Micrococcus luteus can be particularly challenging as Micrococcus luteus has been found to be unstable in aqueous and polar solvents and carriers, and is chemical and heat sensitive. One solution to the viability problem has been two-part formulations where a live microorganism component is mixed with a solvent or carrier component immediately prior to use. Drawbacks to this approach include additional cost and complexity of the manufacturing process, lack of convenience for the user, and additional packaging required. Accordingly, there is still a need for a cosmetic composition containing all components including live, viable Micrococcus luteus Q24, in a single formulation.

A particular problem in working with Micrococcus luteus is the production of compositions with higher viscosity components which require heating or melting in the production of the composition. Heating can kill or adversely affect viability of Micrococcus luteus.

Another problem is the requirement for the formulation to be suitable for maintaining microbiological stability and viability of Micrococcus luteus, and providing good temporal, thermal and physical stability of the formulation.

Consumer demand for preservative-free and/or antiallergenic products presents even further challenges, as the options for formulation ingredients are more limited, increasing the difficulty of providing resultant formulations with desirable properties.

It is an object of the invention to provide Micrococcus luteus compositions which go at least some way towards addressing one or more of the above problems; and/or to at least provide the public with a useful choice.

Other objects of the invention may become apparent from the following description which is given by way of example only.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides a topical composition comprising Micrococcus luteus Q24 and a viscosity modifier, the composition having a viscosity of from about 2,500,000 to about 8,000,000 cP at 25° C.

In a second aspect, the invention provides a topical composition comprising
about 0.5 to 2% w/w Micrococcus luteus Q24,
about 15 to about 35% w/w beeswax,
about 2 to about 10% w/w cetostearyl alcohol,
about 10% to about 30% w/w cocoa butter,
a quantity sufficient amount of oil vehicle,
wherein the composition has a viscosity of from about 2,500,000 to about 8,000,000 CP at 25° C.

In a third aspect, the invention provides a topical composition comprising
about 1×10³ to about 1×10¹² cfu/g Micrococcus luteus Q24,
about 15 to about 35% w/w beeswax,
about 2 to about 10% w/w cetostearyl alcohol,
about 10% to about 30% w/w cocoa butter,
a quantity sufficient amount of oil vehicle,
wherein the composition has a viscosity of from about 2,500,000 to about 8,000,000 CP at 25° C.

In a fourth aspect, the invention provides a topical composition comprising
about 0.5 to 2% w/w Micrococcus luteus Q24,
about 10 to about 30% w/w beeswax,
about 10% to about 30% w/w shea butter,
about 10% to about 30% w/w cocoa butter,
a quantity sufficient amount of oil vehicle,
wherein the composition has a viscosity of from about 2,500,000 to about 8,000,000 cP at 25° C.

In a fifth aspect, the invention provides a topical composition comprising
about 1×10³ to about 1×10¹² cfu/g Micrococcus luteus Q24,
about 10 to about 30% w/w beeswax,
about 10% to about 30% w/w shea butter,
about 10% to about 30% w/w cocoa butter,
a quantity sufficient amount of oil vehicle,
wherein the composition has a viscosity of from about 2,500,000 to about 8,000,000 CP at 25° C.

In a sixth aspect, the invention provides a method to improve appearance of skin or at least one sign of aging comprising applying to the skin a topical composition according to the first, second or third aspect.

In a seventh aspect, the invention provides a topical composition according to the first, second or third aspect for improving the appearance of skin or at least one sign of aging, or reducing skin dryness.

In an eighth aspect, the invention provides a use of a topical composition according to the first, second or third aspect in the manufacture of medicament for improving the appearance of skin or at least one sign of aging, or reducing skin dryness.

In a ninth aspect, the invention provides a method of manufacturing a topical composition comprising Micrococcus luteus Q24 comprising the steps of:
a) heating a mixture of an oil vehicle and a viscosity modifier to provide a melt mixture,
b) cooling the melt mixture to a temperature in the range of about 35° C. to about 28° C.,
c) adding Micrococcus luteus Q24 to the melt mixture from step b) to provide a uniform mixture,
d) cooling the mixture from step c) to room temperature to provide the composition.

In a tenth aspect, the invention provides a method of manufacturing a topical composition comprising Micrococcus luteus Q24 comprising the steps of:
a) heating a mixture of an oil vehicle and a viscosity modifier to provide a melt mixture,
b) cooling the melt mixture to a temperature between about 22° C. to about 27° C.,
c) adding Micrococcus luteus Q24 to the cooled mixture from step b) and mixing to provide a uniform mixture.

The following embodiments and preferences may relate alone or in any combination of any two or more to any of the above aspects.

In various embodiments, the composition comprises Micrococcus luteus Q24 in an amount of about 1×10³ to about 1×10¹² cfu/g.

In various embodiments, the composition comprises one or more viscosity modifiers in a combined amount of about 30 to about 80% w/w.

In various embodiments, the viscosity modifier is selected from the group consisting of hydrophobic silica, hydrophilic silica, white beeswax (cera alba), yellow beeswax (cera alba), ethyl cellulose, emulsifying wax, cocoa butter, shea butter, fatty alcohols, glyceryl monostearate, candelilla wax, lanolin, and a combination of any two or more thereof. In addition to being a viscosity modifier, lanolin functions as a skin protective agent.

In various embodiments, the viscosity modifier is selected from a combination of white beeswax or yellow beeswax, with cetostearyl alcohol.

In various embodiments, the composition comprises a dispersing agent.

In various embodiments, the composition comprises the dispersing agent in an amount of about 0.1 to about 5% w/w.

In various embodiments, the dispersing agent is selected from the group consisting of polysorbate 80, polysorbate 20, sorbitan oleate, egg lecithin, soybean lecithin, polyoxyl 35 castor oil, sodium stearoyl glutamate, sodium cocoyl isethionate, cetearyl olivate, and a combination of any two or more thereof.

In various embodiments, the composition comprises about 0.5 to about 2% w/w polysorbate 80.

In various embodiments, the composition further comprises an oil vehicle.

In various embodiments, the oil vehicle is selected from the group consisting of a medium chain triglyceride (MCT) oil, plant oil, or a combination thereof.

In various embodiments, the MCT oil is a caprylic/capric triglyceride oil.

In various embodiments, the plant oil is selected from the group consisting of sunflower oil, canola oil, soybean oil, olive oil, jojoba oil, argan oil, rosehip oil, marula oil, chamomile oil, tamanu oil, grapeseed oil, calendula oil, pomegranate oil, macadamia oil, buriti fruit oil, sweet almond oil, evening primrose oil, and a combination of any two or more thereof.

In various embodiments, the plant oil is selected from the group consisting of MCT oil, olive oil, calendula oil, pomegranate oil, and a combination of any two or more thereof.

In various embodiments, the plant oil is MCT oil or olive oil.

In various embodiments, Micrococcus luteus Q24 is lyoprotectant-free.

In various embodiments, the composition further comprises one or more additional probiotic(s).

In various embodiments, the additional probiotic(s) is selected from the group consisting of a Streptococcus spp., a Lactobacillus spp., Limosilactobacillus spp., a Lacticaseibacillus spp., a Ligilactobacillus spp., Lactiplantibacillus spp., a Bifidobacterium spp., a Saccharomyces spp., and a combination of any two or more thereof.

In various embodiments, the Streptococcus spp. is selected from the group consisting of Streptococcus salivarius K12, Streptococcus salivarius M18, Streptococcus salivarius 24 SMB, Streptococcus oralis (e.g. S. oralis 89a), Streptococcus salivarius DB-B5, and a combination of any two or more thereof.

In various embodiments, the composition comprises each additional probiotic in an amount of about 1×10³ to about 1×10¹² cfu/g.

In various embodiments, the composition further comprises an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbant or neutraliser, a sunscreen agent, and a combination of any two or more thereof.

In various embodiments, the inhibitory activity enhancer is selected from the group consisting of sodium chloride, ethylenediaminetetraacetic acid, arginine, calcium carbonate, and a combination of any two or more thereof.

In various embodiments, the buffering agent is selected from the group consisting of calcium carbonate, magnesium carbonate, sodium hydrogen phosphate, sodium dihydrogen phosphate, dipotassium hydrogen phosphate, and potassium dihydrogen phosphate, magnesium carbonate, urea, hydrated aluminium oxides, hydrated aluminum oxides, bentonite clays, kaolin clay, and a combination thereof.

In various embodiments, the antibacterial agent is selected from the group consisting of xylitol, erythritol, epidermin, nisin, salivaricin A, salivaricin A1, salivaricin A2, salivaricin B, and a combination thereof. In various embodiments, the antibacterial agent is selected from the group consisting of xylitol, erythritol, epidermin, nisin, salivaricin A, salivaricin A1, salivaricin A2, salivaricin B, salivaricin 9, salivaricin MPS, salivaricin D (Birri et al., Appl Environ Microbiol. 2012 January; 78 (2): 402-410; GenBank accession number JN564797), salivaricin M18, (Wescombe et al., Future Microbiol. 2012; 7 (12): 1355-1371; American Type Culture Collection, Virginia, United States, accession number BAA 2593; Deutsche Sammlung von Mikroorganismen und Zellkulturen GmbH, Braunschweig, Germany, accession number DSM 14685), salivaricin G32 (Wescombe et al., International Journal of Microbiology, vol. 2012, Article ID 738503, 2012; GenBank accession number JN831266), and a combination thereof.

In various embodiments, the prebiotic(s) is selected from the group consisting of Manuka honey powder, olive squalene, pomegranate seed oil, flax seed oil, coconut oil, colloidal oatmeal, vitamin E, vitamin C, retinol (vitamin A), olive oil, hyaluronic acid, calendula oil, almond oil, tomato oil, allantoin, aloe vera powder, colloidal oatmeal, xylitol, yeast extract, fructooligosaccharide powder, fructooligosaccharide liquid, fructooligosaccharides, galactooligosaccharides, xylooligosaccharides, niacinamide, sunscreen, and a combination of any two or more thereof.

In various embodiments, the prebiotic(s) is selected from the group consisting of yeast extract, cysteine, maltodextrin, inulin, liquorice root, liquorice extract, honey, and a combination of any two or more thereof.

In various embodiments, the composition comprises 0.1 to 35% w/w prebiotic(s).

In various embodiments, the prebiotic(s) is selected from olive squalene, pomegranate seed oil, vitamin E, or a combination thereof.

In various embodiments, the prebiotic(s) is selected from a combination of olive squalene and pomegranate seed oil; pomegranate seed oil and oatmeal; olive squalene and vitamin E; and olive squalene and pomegranate seed oil and vitamin E.

In various embodiments, the composition is preservative-free.

In various embodiments, the composition further comprises one or more additional postbiotic(s).

In various embodiments, the postbiotic is a ferment, a filtrate, a supernatant, a ferment filtrate, or a lysate.

In various embodiments, the postbiotic(s) is selected from the group consisting of Streptococcus ferment, Streptococcus ferment filtrate, Streptococcus ferment lysate, Streptococcus lysate, Streptococcus filtrate, Lactobacillus (including Limosilactobacillus spp, Ligilactobacillus spp, and Lactiplantibacillus spp) ferment, Lactobacillus (including Limosilactobacillus spp, Ligilactobacillus spp, and Lactiplantibacillus spp) filtrate, Micrococcus ferment lysate, Bifidobacterium ferment lysate, Bifidobacterium ferment filtrate, Galactomyces ferment filtrate, Saccharomyces ferment filtrate, Bacillus Ferment, Bacillus filtrate, and a combination of any two or more thereof.

In various embodiments, the fragrance is selected from the group consisting of rose water, orange blossom, rose gardenia, peony, white jasmine, ylang ylang oil, geranium oil, rose oil, and a combination of any two or more thereof.

In various embodiments, the antioxidant is selected from the group consisting of vitamin E, resveratrol, squalene, vitamin C, β-carotene, retinyl acetate, retinyl palmitate, retinol, niacinamide, green tea, green tea extract, caffeine, bakuchiol, licorice root, and a combination of any two or more thereof.

In various embodiments the antioxidant is selected from olive squalene, pomegranate seed oil, flax seed oil, coconut oil, colloidal oatmeal, vitamin E, vitamin C, retinol (vitamin A), olive oil, hyaluronic acid, calendula oil, almond oil, tomato oil, and a combination of any two or more thereof.

In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids, beta-hydroxy acids, vitamin B5, seaweed, seaweed extract, silicone, xylitol, oat extract, oatmeal powder, colloidal oatmeal, aloe vera, sunscreen, lactic acid, camellia oil, hemp seed oil, sea buckthorn fruit oil, lanolin, and a combination of any two or more thereof.

In various embodiments, the antimicrobial is selected from the group consisting of zinc, salicylic acid, azelaic acid, benzoyl peroxide, tea tree oil, salicylic acid, and a combination thereof.

In various embodiments, the composition further comprises from about 0.1% to about 10% w/w olive squalene, and from about 0.1 to about 10% w/w pomegranate seed oil.

In various embodiments, the composition further comprises from about 0.1% to about 10% w/w olive squalene, and from about 0.1 to about 3% w/w vitamin E.

In various embodiments, the composition comprises from about 0.1% to about 10% w/w olive squalene, and from about 0.1 to about 10% w/w pomegranate seed oil, and from about 0.1 to about 3% w/w vitamin E.

In various embodiments, improving the appearance of skin or at least one sign of aging includes that skin looks more radiant, skin looks healthier, skin feels more hydrated, pores are reduced in size, skin feels softer, skin looks clearer, wrinkles are reduced, dryness is reduced, spots are reduced, impurities are reduced, moisture is increased, keratin is reduced, and sebum production is modulated.

In various embodiments, improving the appearance of skin includes improving at least one of teething rash, nappy rash, or dry skin in infants.

In various embodiments, improving the appearance of skin or at least one sign of aging includes reducing skin dryness.

In various embodiments, the composition is non-aqueous.

In various embodiments, the composition has a shelf-life of at least 3 months at 25° C., at 60% RH. In various embodiments, the composition has a shelf-life of at least 12 months at 25° C., at 60% RH.

In various embodiments, the oil vehicle is a medium chain triglyceride.

In various embodiments, the composition further comprises 0.1 to 10% w/w prebiotic(s). In various embodiments, the composition further comprises 0.1 to 35% w/w prebiotic(s).

In various embodiments, the prebiotic(s) is selected from olive squalene, pomegranate seed oil, vitamin E, or a combination thereof.

In various embodiments, the composition further comprises 0.1 to 10% w/w postbiotic(s). In various embodiments, the composition further comprises 0.1 to 35% w/w postbiotic(s).

In various embodiments, the prebiotics are selected from a combination of olive squalene and pomegranate seed oil; olive squalene and vitamin E; and olive squalene and pomegranate seed oil and vitamin E.

In various embodiments, the particle size (Dv90) of Micrococcus luteus Q24 is less than about 300 μm.

In various embodiments, the particle size of Micrococcus luteus Q24 is less than about 250 μm, or less than about 100 μm.

In various embodiments of the ninth aspect, the melt mixture is cooled to about 30° C. in step (b).

In various embodiments of the tenth aspect, the melt mixture is cooled to room temperature in step (b).

In various embodiments of the ninth and tenth aspects, the method further comprises adding a dispersing agent during step (a) or before step (b).

In various embodiments of the seventh and eighth aspects, the topical composition manufactured is a composition of the first, second, third, fourth, fifth or seventh aspects.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Although the present invention is broadly as defined above, those persons skilled in the art will appreciate that the invention is not limited thereto and that the invention also includes embodiments of which the following description gives examples.

### BRIEF DESCRIPTION OF THE FIGURES

The present invention will be described with reference to the accompanying figures, in which:

FIG. 1 shows the shelf-life stability of Micrococcus luteus Q24 freeze-dried raw ingredient under refrigerated storage condition of 4° C. P1: Trehalose as lyoprotectant. P2: Blis Technologies Ltd, trimix blend of Trehalose, Maltodextrin and lactitol as lyoprotectant. P3: No lyoprotectant.

FIG. 2 shows Stability Data for balm formulations at 25° C.±2° C./60% RH over 3 months.

FIG. 3 shows shows microbiological cell counts of a deodorant stick disclosed in WO2006/104403 under controlled storage conditions (25° C./60% RH), over a period of 4 months.

FIG. 4 shows spreadability (Percentage increase in Diameter) of BLT-BB5, BLT-BB9 and BLT-BB2.

FIG. 5A shows Cumulative Syneresis graphed at different Centrifuge speeds (1000, 1500, 5000 and 13000 rpm) at the 20-minute time point for selected formulations shown in Table 1 and the deodorant stick prepared according to example 2 of WO 2006/104403 (trace “deodorant stick”). Cumulative syneresis includes oil leakage (%) released at all time points prior to and including at 20 minutes.

FIG. 5B shows Cumulative Syneresis graphed at different Centrifuge speeds (1000, 1500, 5000 and 13000 rpm) at the 20-minute time point for serum formulations containing 4, 5, 6 and 7% hydrophobic silica shown in Table 4.

FIG. 6 shows a scatter plot showing the viabilities (relative to the PBS negative control treatment) of the individual tissues given each dose level of Micrococcus luteus Q24. Treatment groups 5-9 were given Micrococcus luteus Q24 at doses of 10₅-10₉ cfu/mL, respectively. The thick horizontal bars show group means and the error bars indicate 95% confidence intervals *, significant difference with P<0.05.

FIG. 7 shows a scatter plot of individual IL-6, IL 8 and IL-18 levels measured in the conditioned media of the tissues on days 0 and 4. Thick horizontal bars are group means and the error bars show the 95% confidence intervals. **, significantly different with P<0.01.

FIG. 8 shows histology of EpiDerm treated for 5 days with Micrococcus luteus Q24 (and PBS and vitamin C-data not shown). Sections were stained with hematoxylin and eosin stain (H&E) and were imaged at 40× magnification. Arrow “B” points to the permeable membrane on which tissues were grown. Tissue layers shown are “C” basal layer, “D” granular layer, “E” stratum corneum. Arrows labelled “A” show nuclear remnants caught in stratum corneum (presence indicates possible acceleration of stratum corneum production).

FIG. 9 shows a scatter plot of individual changes in IL-6, IL-8, and IL-18 levels measured on days 4 and 5 in the conditioned media of the tissues given combinations of main treatments and +0.5% sodium dodecyl sulphate (SDS). Thick horizontal bars are group means and the error bars show the 95% confidence intervals. ***, significantly different with P<0.001.

FIG. 10 shows scores given by participants to various sensory properties of balm formulations BB9, BB14, BB15 and BB18, in the Balm Sensory Trial of Example 9.

FIG. 11 shows the overall score of balms BB9, BB14, BB15 and BB18 in the Balm Sensory Trial of Example 9.

FIG. 12 shows percentage change in skin parameters, as analyzed using a Dermo Prime skin analyser device (DermoPrime Viso, CHOWIS, South Korea) and associated software, for trial partcipants using Balm A (main area/forearm, top left; area of concern, bottom left) and Balm B (main area/forearm, top right; area of concern, bottom right) in the seven-day Sensory and Efficacy Trial of Example 10.

FIG. 13 shows percentage change in skin parameters on the Area of Concern (Balm A), as analyzed using a Dermo-Prime Viso skin analyser device and software in the Sensory and Efficacy Trial of Example 10. p-values are calculated (95% confidence) using a T-test (Paired Two Sample for Means) for this data.

FIG. 14 shows percentage change in skin parameters on the Area of Concern (Balm B), as analyzed using a Dermo-Prime Viso skin analyser device and software in the Sensory and Efficacy Trial of Example 10. p-values are calculated (95% confidence) using a T-test (Paired Two Sample for Means) for this data.

FIG. 15 shows percentage change in moisture skin parameter (left) and keratin parameter (right) where participants are grouped by age: Younger age group (Group A, mean age 21-32 y) vs Middle age (38-50 y). Statistically significant differences were observed for both age groups, however, more significant improvement (moisture content and keratin) was observed in the middle-aged group versus the younger group for both balms applied to the areas of concern suggesting anti-ageing benefits of the balms. p-values are calculated (95% confidence) using a T-test (Paired Two Sample for Means)

FIG. 16 shows growth curves of Micrococcus luteus Q24 with potential prebiotics.

FIG. 17 shows growth curves of combinations of prebiotics-olive squalene and pomegranate seed oil; olive squalene and oatmeal flour (colloidal oatmeal); and olive squalene and vitamin E.

### DETAILED DESCRIPTION OF THE INVENTION

### Definitions

The term “comprising” as used in this specification and claims means “consisting at least in part of”. When interpreting each statement in this specification and claims that includes the term “comprising”, features other than that or those prefaced by the term may also be present. Related terms such as “comprise”, “comprised” and “comprises” are to be interpreted in the same manner.

As used herein the term “and/or” means “and” or “or”, or both.

As used herein “(s)” following a noun means the plural and/or singular forms of the noun.

The general chemical and biological terms used, for example, in the formulae herein have their usual meanings.

The terms “composition” and “formulation” are used interchangeably herein. The term “balm” as used herein refers to a semisolid topical composition which melts when it comes into contact with skin. The balm formulations of the invention have a viscosity higher than a serum or a cream, with the viscosity of the balms being in the range of from about 2,500,000 to about 8,000,000 cP at 25° C. The term “balm” as used herein does not include hard lip-balm sticks which have a viscosity higher than this range, typically in excess of 9,000,000 cP at 25° C.

The term “platform” is used herein to refer to a group of balm formulations having a similar combination of viscosity modifier and oil vehicle.

The term “subject” as used herein refers to a mammal, including humans, dogs, cats, horses, sheep, cows and other domestic and farm animals.

A postbiotic is defined as a preparation of inanimate microorganisms and/or their components that confers a health benefit on the host (Salminen, S., Collado, M. C., Endo, A. et al. The International Scientific Association of Probiotics and Prebiotics (ISAPP) consensus statement on the definition and scope of postbiotics. Nat Rev Gastroenterol Hepato/18, 649-667 (2021). Postbiotics suitable for use in the invention are discussed below.

The term “self-emulsifying glyceryl monostearate” is used herein to refer to compositions which comply with the requirements of the BP monograph (2021) “Self-emulsifying Glyceryl Monostearate”. IMWITOR® 960 K (IOI Oleo GmbH) is an ester of palmitic and stearic fatty acid with glycerol. Fatty acids and glycerol are derived from vegetable sources. It contains at least 30.0% of monoglycerides and additionally approx. 5% of alkaline stearate. IMWITOR® 960 K fully complies with the requirements of the current B.P. monograph “Self-emulsifying Glyceryl Monostearate”.

The unit “cfu/g” means colony-forming units per gram. A colony-forming unit (CFU) is a unit used to estimate the number of viable bacteria in a sample. Determining colony-forming units requires culturing the microbes and counts only viable cells on a nutrient agar medium, i.e., those able to multiply and form a visible colony.

The unit “% w/w” means the percentage weight based on the total weight of the composition.

As used herein, “room temperature” refers to a temperature of 25° C.

The term “lyoprotectant-free” or “cryoprotectant-free” as used herein means Micrococcus luteus has been produced in the absence of a lyoprotectant or cryoprotectant, or both.

The term “preservative-free” is used herein to refer to a composition which has been prepared without adding preservatives.

The term “uniform” is used herein to refer to a composition which is homogeneous throughout. Uniformity of the composition can be achieved by mixing the composition using a range of methods, including stirring by hand or automated stirring or mechanized stirring, including use of a magnetic stirrer bar or use of a homogenizer.

The term “improving the appearance of skin or at least one sign of aging in a subject” as used herein means an improvement in at least one parameter commonly used for skin analysis including skin radiance, skin health, skin hydration, pore size, skin softness, skin clarity, moisture levels, sebum levels, appearance of wrinkles, dryness, roughness, dullness, appearance of spots including age spots, and impurities.

### Topical Composition

The invention provides a topical composition comprising Micrococcus luteus Q24 and a viscosity modifier, the composition having a viscosity of from about 2,500,000 to about 8,000,000 cP at 25° C.

The invention also provides a topical composition comprising
about 0.5 to 2% w/w Micrococcus luteus Q24,
about 15 to about 35% w/w beeswax,
about 2 to about 10% w/w cetostearyl alcohol,
about 10% to about 30% w/w cocoa butter,
a quantity sufficient amount of oil vehicle,
wherein the composition has a viscosity of from about 2,500,000 to about 8,000,000 CP at 25° C.

The invention also provides a topical composition comprising
about 1×10³ to about 1×10¹² cfu/g Micrococcus luteus Q24,
about 15 to about 35% w/w beeswax,
about 2 to about 10% w/w cetostearyl alcohol,
about 10% to about 30% w/w cocoa butter,
a quantity sufficient amount of oil vehicle,
wherein the composition has a viscosity of from about 2,500,000 to about 8,000,000 CP at 25° C.

The invention also provides a topical composition comprising
about 0.5 to 2% w/w Micrococcus luteus Q24,
about 10 to about 30% w/w beeswax,
about 10% to about 30% w/w shea butter,
about 10% to about 30% w/w cocoa butter,
a quantity sufficient amount of oil vehicle,
wherein the composition has a viscosity of from about 2,500,000 to about 8,000,000 CP at 25° C.

The invention also provides a topical composition comprising
about 1×10³ to about 1×10¹² cfu/g Micrococcus luteus Q24,
about 10 to about 30% w/w beeswax,
about 10% to about 30% w/w shea butter,
about 10% to about 30% w/w cocoa butter,
a quantity sufficient amount of oil vehicle,
wherein the composition has a viscosity of from about 2,500,000 to about 8,000,000 CP at 25° C.

In various embodiments, the composition further comprises about 0.5 to about 2% w/w polysorbate 80.

In various embodiments, the oil vehicle is a medium chain triglyceride oil, or olive oil.

In various embodiments, the composition is preservative-free. For context, preservatives typically used in prior art skincare products include antioxidants and antimicrobials such as parabens, benzoates, sorbates, and sulphites. Some consumers exhibit allergic reactions to preservative compounds, or prefer to use natural products. The compositions of the present invention advantageously do not require preservatives to prevent microbial contamination.

The topical composition described herein may be used for improving the appearance of skin or at least one sign of aging. In various embodiments, the topical composition may be used for improving the hydration of skin or decreasing skin dryness.

Described herein is the use of a composition comprising Micrococcus luteus Q24 and a viscosity modifier, the composition having a viscosity of from about 2,500,000 to about 8,000,000 cP at 25° C., in the manufacture of medicament for improving the appearance of skin or at least one sign of aging.

Micrococcus luteus Q24 useful in the present invention has been found by a tissue culture model to be well tolerated, to not elicit an anti-inflammatory response, to have an anti-inflammatory effect, to accelerate the growth of stratum corneum which may help in rejuvenating and hydrating the skin, and reducing pores and wrinkles in skin (see example 8). The Micrococcus luteus Q24 compositions described herein are also shown to have beneficial effects on skin quality parameters, notably including increase of moisture and decrease of keratin (see example 10). The compositions described herein are thus also useful for improving the appearance of skin, including dry skin, in infants, such as teething rash on the mouth, chin, neck or chest from saliva, and nappy rash from stool or urine.

### Methods of Use

Described herein is a method to improve appearance of skin or at least one sign of aging comprising applying to the skin a topical composition comprising Micrococcus luteus Q24 and a viscosity modifier, the composition having a viscosity of from about 2,500,000 to about 8,000,000 cP at 25° C. Also described herein is a topical composition comprising Micrococcus luteus Q24 and a viscosity modifier, the composition having a viscosity of from about 2,500,000 to about 8,000,000 cP at 25° C., for improving the appearance of skin or at least one sign of aging. Further described herein is use of a topical composition comprising Micrococcus luteus Q24 and a viscosity modifier, the composition having a viscosity of from about 2,500,000 to about 8,000,000 cP at 25° C. in the manufacture of medicament for improving the appearance of skin or at least one sign of aging.

Micrococcus luteus Q24

Micrococcus luteus is a normal bacterial member (commensal) on human skin and is a key bacterium in keeping the balance among the various microbial flora of the skin.

Micrococcus luteus Q24 was deposited with Deutsche Sammlung von Mikro organisms Und Zellkulturen GmbH, Braunschweig, Germany, on 10 Mar. 2005, and assigned accession number DSM 17172. Micrococcus luteus strain Q24 is described in WO2006104403, incorporated herein by reference.

In various embodiments, the Micrococcus luteus is a live probiotic.

In various embodiments, the composition of the invention, which is useful in the method of the invention, comprises Micrococcus luteus Q24 in an amount of about 1×10³ to about 1×10¹² cfu/g. In various embodiments, the composition comprises Micrococcus luteus Q24 in an amount of about 1×10⁴ to about 1×10¹², about 1×10⁵ to about 1×10¹², about 1×10⁶ to about 1×10¹², about 1×10⁷ to about 1×10¹², about 1×10⁸ to about 1×10¹², about 1×10⁴ to about 1×10¹⁰, about 1×10⁵ to about 1×10¹⁰, about 1×10⁶ to about 1×10¹⁰, about 1×10⁷ to about 1×10¹⁰, about 1×10⁸ to about 1×10¹⁰, about 1×10⁴ to about 1×10⁹, about 1×10⁵ to about 1×10⁹, about 1×10⁶ to about 1×10⁹, about 1×10⁷ to about 1×10⁹ cfu/g. In various embodiments, the composition comprises Micrococcus luteus Q24 in an amount of about 1×10⁹ cfu/g.

In various embodiments, Micrococcus luteus Q24 is freeze-dried or lyophilized. In various embodiments, Micrococcus luteus Q24 is provided in a lyoprotectant or cryoprotectant. Lyoprotectants and cryoprotectants are commonly used in the manufacture of products containing probiotics to protect and maintain cell viability. The terms “lyoprotectant” and “cryoprotectant” refer to compositions that protect active ingredients, in this case, Micrococcus luteus Q24. Lyoprotectants protect during drying, while cryoprotectants protect during freezing. The same composition can have both functions, and unless otherwise specified, the terms are used interchangeably herein.

Suitable lyoprotectants or cryoprotectants will be known to a person skilled in the art. In various embodiments, the lyoprotectant is trehalose, or a trimix comprising trehalose, maltodextrin, and lactitol.

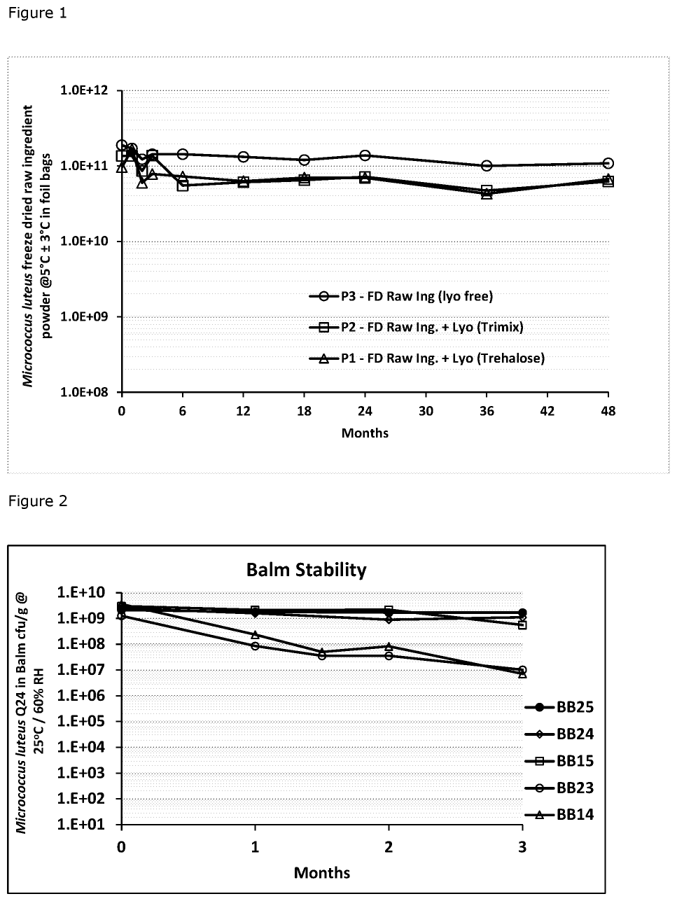

Surprisingly, the applicants have identified that Micrococcus luteus Q24 can also be freeze-dried (FD) in the absence of lyoprotectants or cryoprotectants with no significant loss in cell viability as shown in FIG. 1. This result is counter-intuitive, where microorganisms routinely need to be protected during the freeze-drying process. In various embodiments, Micrococcus luteus Q24 is lyoprotectant-free in the composition.

As will be appreciated, this finding leads to significant manufacturing advantages where the freeze-drying process is simplified, and costs are reduced where no lyoprotectants or cryoprotectants need to be used.

### Viscosity Modifier

The composition comprises one or more viscosity modifiers to bring the viscosity within the range of from about 2,500,000 to about 8,000,000 cP at 25° C. Compositions with a viscosity in this range can be formulated as a balm composition. Such balm compositions can support the live probiotic over a long residence time on the skin. Balm formulations containing live Micrococcus luteus Q24 have not been described in the prior art. For example, the hard deodorant stick of WO 2006/104403 has a very high viscosity which is outside this range and outside the range of the measurement equipment used by the inventors (greater than 9,375,000 cP at 25° C.). While not measured, the saline suspension of WO 2006/104403 is expected to have a viscosity comparable to that of water (about 1 cP at 25° C.).

In various embodiments, the composition comprises one or more viscosity modifiers in a combined amount of about of about 30 to about 80% w/w. For example, the composition may comprise the viscosity modifier in an amount of about 30 to about 70%, or about 30 to about 60%, or about 30 to about 55%, or about 40 to about 80%, or about 40 to about 70%, or about 40 to about 60%, or about 40 to about 55%, or about 45 to about 55% w/w. For example, the composition may comprise the viscosity modifier in an amount of about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79% or about 80% w/w based on the total weight of the composition. In various embodiments, the composition comprises the one or more viscosity modifiers (e.g, a combination of white beeswax or yellow beeswax, with cetostearyl alcohol) in an amount of about 50% w/w.

Suitable viscosity modifiers include, but are not limited to, hydrophobic silica, hydrophilic silica, white beeswax, yellow beeswax, paraffin wax, jojoba wax, microcrystalline wax, ethyl cellulose, emulsifying wax, stearic acid, xanthan gum, tapioca starch, Carbopol polymer (e.g. 971p, 974p), cocoa butter, shea butter, fatty alcohols, self-emulsifying glyceryl monostearate, candelilla wax, lanolin, and a combination of any two or more thereof. In a preferred embodiment, the viscosity modifier is selected from a combination of white beeswax or yellow beeswax, with cetostearyl alcohol.

In some embodiments, the viscosity modifier comprises a fatty alcohol, such as cetyl alcohol, stearyl alcohol, or cetostearyl alcohol. In a preferred embodiment, the viscosity modifier comprises cetostearyl alcohol. Cetostearyl alcohol is also known as cetearyl alcohol or cetylstearyl alcohol, and is a mixture of the fatty alcohols cetyl and stearyl alcohol. The inventors have found that balm formulations comprising cetostearyl alcohol exhibit good spreadability and sensory properties. This is discussed further below.

In some embodiments, the viscosity modifier comprises a combination of self-emulsifying glyceryl monostearate, shea butter and cocoa butter. In various embodiments, the composition comprises self-emulsifying glyceryl monostearate, shea butter and cocoa butter in a combined amount of about of about 30 to about 80% w/w, for example a combined amount of about 30 to about 70%, or about 40 to about 60%, or about 45 to about 55% w/w. In various embodiments the composition comprises self-emulsifying glyceryl monostearate in an amount of about 5 to about 30% w/w, shea butter in an amount of about 10% to about 35% w/w, and cocoa butter in an amount of about 10% to about 35% w/w.

In some embodiments, the viscosity modifier comprises a combination of white or yellow beeswax, cetostearyl alcohol and cocoa butter. In various embodiments, the composition comprises white or yellow beeswax, cetostearyl alcohol and cocoa butter in a combined amount of about of about 30 to about 80% w/w, for example a combined amount of about 30 to about 70%, or about 40 to about 60%, or about 45 to about 55% w/w. In various embodiments the composition comprises white or yellow beeswax in an amount of about 15 to about 35% w/w, cetostearyl alcohol in an amount of about 2 to about 10% w/w, and cocoa butter in an amount of about 10% to about 30% w/w.

In some embodiments, the viscosity modifier comprises a combination of white or yellow beeswax, shea butter and cocoa butter. In various embodiments, the composition comprises white or yellow beeswax, shea butter and cocoa butter in a combined amount of about of about 30 to about 80% w/w, for example a combined amount of about 30 to about 70%, or about 40 to about 60%, or about 45 to about 55% w/w. In various embodiments the composition comprises white or yellow beeswax in an amount of about 15 to about 35% w/w, shea butter in an amount of about 10% to about 30% w/w, and cocoa butter in an amount of about 10% to about 30% w/w.

### Dispersing Agent

In various embodiments, the composition comprises a dispersing agent. Advantageously, the dispersing agent may facilitate dispersion of the solid particles in the composition. For example, the dispersing agent may facilitate dispersion of the probiotic in the composition. Additionally, the dispersing agent may assist in emulsification of the oil phase with aqueous phase when mixed together before application. The dispersing agent may be a non-ionic dispersing agent or an amphoteric dispersing agent. Examples of non-ionic dispersing agents include, but are not limited to polysorbate 80 (Tween 80), polysorbate 20 (Tween 20), sorbitan oleate (Span 80), polyoxyl 35 castor oil (Cremaphor EL), cetearyl olivate. Examples of amphoteric dispersing agents include, but are not limited to, lecithin, such as egg lecithin and soybean lecithin, sodium stearoyl glutamate, sodium cocoyl isethionate.

In various embodiments, the composition comprises the dispersing agent in an amount of about 0.1 to about 5% w/w. For example, the composition may comprise the dispersing agent in an amount of about 0.5 to about 4%, about 0.5 to about 3%, about 0.5 to about 2.5% w/w, about 0.5 to about 2% w/w or about 1 to about 2% w/w. In various embodiments, the composition comprises the dispersing agent (e.g. Tween 80) in an amount of about 1% w/w, or about 2% w/w.

### Oil Vehicle

In various embodiments, the composition comprises an oil vehicle. Suitable oil vehicles include, but are not limited to, medium chain triglycerides (MCT) and plant oils. Preferably, the medium chain triglyceride is a caprylic/capric triglyceride, such as Miglyol 812N (triglyceride ester of saturated coconut/palm-kernel oil derived caprylic and capric fatty acids and plant derived glycerol). In various embodiments, the plant oil is selected from the group consisting of sunflower oil, canola oil, soybean oil, olive oil, jojoba oil, argan oil, rosehip oil, marula oil, chamomile oil, tamanu oil, grapeseed oil, calendula oil, pomegranate oil, macadamia oil, buriti fruit oil (available from e.g. Paris Fragrances (USA), sweet almond oil, evening primrose oil, and a combination of any two or more thereof. In some embodiments the oil is MCT oil, olive oil, or a combination thereof.

In various embodiments, the composition comprises the oil vehicle in a quantity sufficient (q.s.) amount, i.e, an amount to bring the total % w/w of the composition to 100%. In various embodiments, the composition comprises the oil vehicle in an amount of about 20 to about 70% w/w. For example, the composition may comprise the oil vehicle in an amount of about 30 to about 70%, or about 30 to about 60%, or about 30 to about 55%, or about 40 to about 70%, or about 40 to about 60%, or about 40 to about 55%, or about 45 to about 55% w/w. For example, the composition may comprise the oil vehicle in an amount of about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70% w/w. In various embodiments, the composition comprises the oil vehicle in an amount of 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63% w/w.

The combination of viscosity modifier(s) and oil vehicle described herein advantageously provides a balm formulation suitable for maintaining microbiological stability and viability of Micrococcus luteus Q24, and providing good temporal, thermal and physical stability of the formulation.

In various embodiments, the composition is non-aqueous. In various embodiments, the composition is substantially anhydrous. In various embodiments, the composition comprises less than 7% water, less than 5% water, less than 3% water, less than 2% water, less than 1% water, less than 0.5% water, less than 0.1% water, or less than 0.01% water. In the present composition, water includes absorbed moisture from the environment.

### Additional Probiotics

In various embodiments, the composition further comprises one or more additional probiotics. Suitable additional probiotics include, but are not limited to, Lactobacillus spp. (e.g. L, acidophilus), Limosilactobacillus spp. (e.g. L. reuteri, previously Lactobacillus reuteri), Lacticaseibacillus spp. (e.g. L. rhamnosus, previously Lactobacillus rhamnosus), Ligilactobacillus spp. (e.g. L. salivarius, previously Lactobacillus salivarius), Lactiplantibacillus spp. (e.g. L. plantarum, previously Lactobacillus plantarum), Bifidobacterium spp. (e.g. B. bifidum, B. longum, or B. lactis BB12), Streptococcus spp. (e.g. S. oralis, S. oralis 89a, S. uberis, S. salivarius 24SMB, S. salivarius M18, S. salivarius K12, or S. salivarius DB-B5), and Saccharomyces spp. (e.g. S. boulardii or S. cerevisiae).

S. salivarius K12 was deposited with Deutsche Sammlung von Mikro organismen Und Zellkulturen GmbH, Mascheroder Weg 1 b, D-38124, Braunschweig, Germany on 8 Oct. 1999, and assigned Accession Nos. DSM 13084. S. salivarius M18 was deposited at Deutsche Sammlung von Mikro organismen Und Zellkulturen GmbH, Mascheroder Weg 1 b, D-38124, Braunschweig, Germany on Dec. 12, 2001, and assigned Accession No. DSM 14685.

In various embodiments, the Streptococcus spp. is selected from the group consisting of Streptococcus salivarius K12, Streptococcus salivarius M18, Streptococcus oralis (e.g. S. oralis 89a), Streptococcus salivarius 24SMB, and a combination of any two or more thereof. In various embodiments, the Streptococcus spp. is selected from the group consisting of Streptococcus salivarius K12, Streptococcus salivarius M18, Streptococcus salivarius 24 SMB, Streptococcus oralis (e.g. S. oralis 89a), Streptococcus salivarius DB-B5, and a combination of any two or more thereof.

In various embodiments, the composition comprises each additional probiotic in an amount of about 1×10³ to about 1×10²² cfu/g. For example, the composition comprises each additional probiotic in an amount of about 1×10⁴ to about 1×10¹², about 1×10⁵ to about 1×10¹², about 1×10⁶ to about 1×10¹², about 1×10⁷ to about 1×10¹², about 1×10⁸ to about 1×10¹², about 1×10⁴ to about 1×10¹⁰, about 1×10⁵ to about 1×10¹⁰, about 1×10⁶ to about 1×10¹⁰, about 1×10⁷ to about 1×10¹⁰, about 1×10⁸ to about 1×10¹⁰, about 1×10⁴ to about 1×10⁹, about 1×10⁵ to about 1×10⁹, about 1×10⁶ to about 1×10⁹, about 1×10⁷ to about 1×10⁹ cfu/g. In various embodiments, the composition comprises each additional probiotic in an amount of about 1×10⁹ cfu/g.

### Postbiotics

In various embodiments, the composition comprises a postbiotic. Postbiotics suitable for use in the invention include ferments, filtrates, lysates, and supernatants. In various embodiments, the postbiotic is selected from the group consisting of a ferment, a filtrate, a supernatant, a ferment filtrate, or a lysate selected from the group consisting of Streptococcus ferment, Streptococcus ferment filtrate, Streptococcus ferment lysate, Streptococcus lysate, Streptococcus filtrate, Lactobacillus (including Limosilactobacillus spp, Ligilactobacillus spp, and Lactiplantibacillus spp) ferment, Lactobacillus (including Limosilactobacillus spp, Ligilactobacillus spp, and Lactiplantibacillus spp) filtrate, Micrococcus ferment lysate, Bifidobacterium ferment lysate, Bifidobacterium ferment filtrate, Galactomyces ferment filtrate, Saccharomyces ferment filtrate, Bacillus Ferment, Bacillus filtrate, and a combination of any two or more thereof. The postbiotic can be in any suitable form including liquids, or powders e.g. freeze-dried or spray-dried powder.

In various embodiments, the composition comprises about 0.1% to about 35% w/w postbiotic, for example from about 10% to about 15% w/w postbiotic.

In various embodiments, the composition comprises a liquid postbiotic in an amount of from about 0.1% to about 30% w/w, for example from about 10% to about 15% w/w.

In various embodiments, the composition comprises a powder postbiotic in an amount of from about 0.1% to about 20%, for example from about 10% to about 15% w/w.

### Additional Additives

Those persons skilled in the art will appreciate the topical composition may comprise other additives conventionally used in a topical composition, such as a moisturiser. Art skilled readers will further appreciate that additives need to be compatible with probiotic viability and efficacy. Such additives may provide or improve a therapeutic, cosmetic, stability, and/or appearance property of the composition. Examples of suitable additives include, but are not limited to, an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbant or neutraliser, or a sunscreen agent. Such additives may be included in the composition of the invention in amounts typical for topical formulations. A variety of pharmaceutically acceptable additives suitable for topical application of viable or lyophilized bacteria are well known in the art. A skilled worker will appreciate that any additional additive should not be inhibitory towards Micrococcus luteus Q24.

A skilled worker will also appreciate that some additives may have dual function, for example, an antioxidant or skin protective agent may also function as a prebiotic.

In various embodiments, the composition further comprises an inhibitory activity enhancer, a buffering agent, an antibacterial agent, a prebiotic, a postbiotic, a fragrance, an antioxidant, a colourant, a skin protective agent, an antimicrobial, an aluminium salt, a mineral pigment, an odour absorbant or neutraliser, a sunscreen agent, and a combination of any two or more thereof.

In various embodiments, the inhibitory activity enhancer is selected from the group consisting of sodium chloride, ethylenediaminetetraacetic acid, arginine, calcium carbonate, and a combination of any two or more thereof.

In various embodiments, the buffering agent is selected from the group consisting of calcium carbonate, magnesium carbonate, sodium hydrogen phosphate, sodium dihydrogen phosphate, dipotassium hydrogen phosphate, potassium dihydrogen phosphate, magnesium carbonate, urea, hydrated aluminium oxide, bentonite clay, kaolin clay, and a combination thereof.

In various embodiments, the antibacterial agent is selected from the group consisting of xylitol, erythritol, epidermin, nisin, salivaricin A, salivaricin A1, salivaricin A2, salivaricin B, and a combination thereof. In various embodiments, the antibacterial agent is selected from the group consisting of xylitol, erythritol, epidermin, nisin, salivaricin A, salivaricin A1, salivaricin A2, salivaricin B, salivaricin 9, salivaricin MPS, and a combination thereof.

In various embodiments, the composition comprises a prebiotic. Prebiotics may enhance the growth of Q24, enhance antimicrobial activity, or enhance the production of compounds to enhance skin quality parameters.

In various embodiments, the prebiotic is selected from the group consisting of Manuka honey, olive squalene, pomegranate seed oil, flax seed oil, coconut oil, colloidal oatmeal, vitamin E, vitamin C, olive oil, hyaluronic acid, calendula oil, almond oil, tomato oil, allantoin, aloe vera powder, inulin (e.g. inulin from Chichorium intybus (chicori) root), Borago officinalis (borage) seed oil, Punica granatum (pomegranate) extract, Siraitia grosvenorii (monk fruit) extract, colloidal oatmeal, xylitol, yeast extract, fructooligosaccharide powder, fructooligosaccharide liquid, fructooligosaccharides, galactooligosaccharides, xylooligosaccharides, and a combination of any two or more thereof.

In various embodiments, the prebiotic is selected from the group consisting of yeast extract, cysteine, maltodextrin, inulin (e.g. inulin from chicori root), liquorice root, liquorice extract, honey, and a combination of any two or more thereof. In various embodiments, the prebiotic is selected from the group consisting of yeast extract, cysteine, maltodextrin, inulin (e.g. inulin from chicori root), liquorice root, liquorice extract, and a combination of any two or more thereof.

In various embodiments, the prebiotic may be an oil prebiotic or a powder prebiotic. In various embodiments, the composition comprises an oil prebiotic and/or a powder prebiotic. In various embodiments, the oil prebiotic is selected from the group consisting of olive squalene, pomegranate seed oil, flax seed oil, coconut oil, vitamin E, olive oil, calendula oil, almond oil, tomato oil, fructooligosaccharide liquid, and a combination of any two or more thereof. In various embodiments, the powder prebiotic is selected from the group consisting of Manuka honey, colloidal oatmeal, vitamin C, hyaluronic acid, allantoin, aloe vera powder, colloidal oatmeal, xylitol, yeast extract, fructooligosaccharide powder, fructooligosaccharides, galactooligosaccharides, xylooligosaccharides, and a combination of any two or more thereof.

In various embodiments, the prebiotic is a combination of olive squalene and pomegranate seed oil; olive squalene and vitamin E; or olive squalene, pomegranate seed oil and vitamin E. The inventors have surprisingly identified that combinations of olive squalene and pomegranate seed oil or olive squalene and vitamin E have a synergistic effect on the growth of Micrococcus luteus Q24.

In various embodiments, the composition comprises about 0.1% to about 10% w/w prebiotic, for example from about 0.1% to about 8%, or about 0.1% to about 6%, or about 0.1% to about 5%, or about 0.3% to about 8%, or about 0.3% to about 6%, or about 0.3% to about 5%, or about 0.5% to about 8%, or about 0.5% to about 6%, or about 0.5% to about 5%, or about 1% to about 8%, or about 1% to about 6%, or about 1% to about 5% w/w prebiotic.

In various embodiments, the composition comprises about 0.1% to about 35% w/w prebiotic, for example from about 0.1% to about 30%, about 0.1% to about 25%, or about 0.1% to about 20%, or about 0.1% to about 18%, or about 0.1% to about 16%, or about 0.1% to about 15%, or about 0.1% to about 12%, or about 0.1% to about 10%, or about 0.1% to about 8%, or about 0.1% to about 6%, or about 0.1% to about 5%, or about 0.1% to about 2%, or about 0.3% to about 35%, or about 0.3% to about 30%, or about 0.3% to about 25%, or about 0.3% to about 20%, or about 0.3% to about 18%, or about 0.3% to about 16%, or about 0.3% to about 15%, or about 0.3% to about 12%, or about 0.3% to about 10%, or about 0.3% to about 8%, or about 0.3% to about 6%, or about 0.3% to about 5%, or about 0.3% to about 2%, or about 0.5% to about 35%, or about 0.5% to about 30%, or about 0.5% to about 25%, or about 0.5% to about 20%, or about 0.5% to about 18%, or about 0.5% to about 16%, or about 0.5% to about 15%, or about 0.5% to about 12%, or about 0.5% to about 10%, or about 0.5% to about 8%, or about 0.5% to about 6%, or about 0.5% to about 5%, or about 0.5% to about 2%, or about 0.5% to about 35%, or about 1% to about 30%, or about 1% to about 25%, or about 1% to about 20%, or about 1% to about 18%, or about 1% to about 16%, or about 1% to about 15%, or about 1% to about 12%, or about 1% to about 10%, or about 1% to about 8%, or about 1% to about 6%, or about 1% to about 5%, or about 0.5% to about 35%, or about 3% to about 30%, or about 3% to about 25%, or about 3% to about 20%, or about 3% to about 18%, or about 3% to about 16%, or about 3% to about 15%, or about 3% to about 12%, or about 3% to about 10%, or about 3% to about 8%, or about 3% to about 6%, or about 3% to about 5%, or about 4% to about 30%, or about 4% to about 25%, or about 4% to about 20%, or about 4% to about 18%, or about 4% to about 16%, or about 4% to about 15%, or about 4% to about 12%, or about 4% to about 10%, or about 4% to about 8%, or about 4% to about 6%, or about 4% to about 5% w/w prebiotic.

In various embodiments, the composition comprises an oil prebiotic in an amount of about 35% w/w prebiotic, for example from about 0.1% to about 30%, about 0.1% to about 25%, or about 0.1% to about 20%, or about 0.1% to about 18%, or about 0.1% to about 16%, or about 0.1% to about 15%, or about 0.1% to about 12%, or about 0.1% to about 10%, or about 0.1% to about 8%, or about 0.1% to about 6%, or about 0.1% to about 5%, or about 0.1% to about 2%, or about 0.3% to about 35%, or about 0.3% to about 30%, or about 0.3% to about 25%, or about 0.3% to about 20%, or about 0.3% to about 18%, or about 0.3% to about 16%, or about 0.3% to about 15%, or about 0.3% to about 12%, or about 0.3% to about 10%, or about 0.3% to about 8%, or about 0.3% to about 6%, or about 0.3% to about 5%, or about 0.3% to about 2%, or about 0.5% to about 35%, or about 0.5% to about 30%, or about 0.5% to about 25%, or about 0.5% to about 20%, or about 0.5% to about 18%, or about 0.5% to about 16%, or about 0.5% to about 15%, or about 0.5% to about 12%, or about 0.5% to about 10%, or about 0.5% to about 8%, or about 0.5% to about 6%, or about 0.5% to about 5%, or about 0.5% to about 2%, or about 0.5% to about 35%, or about 1% to about 30%, or about 1% to about 25%, or about 1% to about 20%, or about 1% to about 18%, or about 1% to about 16%, or about 1% to about 15%, or about 1% to about 12%, or about 1% to about 10%, or about 1% to about 8%, or about 1% to about 6%, or about 1% to about 5%, or about 0.5% to about 35%, or about 3% to about 30%, or about 3% to about 25%, or about 3% to about 20%, or about 3% to about 18%, or about 3% to about 16%, or about 3% to about 15%, or about 3% to about 12%, or about 3% to about 10%, or about 3% to about 8%, or about 3% to about 6%, or about 3% to about 5%, or about 4% to about 30%, or about 4% to about 25%, or about 4% to about 20%, or about 4% to about 18%, or about 4% to about 16%, or about 4% to about 15%, or about 4% to about 12%, or about 4% to about 10%, or about 4% to about 8%, or about 4% to about 6%, or about 4% to about 5% oil prebiotic.

In various embodiments, the composition comprises a powder prebiotic in an amount of about 0.1% to about 20%, or about 0.1% to about 18%, or about 0.1% to about 16%, or about 0.1% to about 15%, or about 0.1% to about 12%, or about 0.1% to about 10%, or about 0.1% to about 8%, or about 0.1% to about 6%, or about 0.1% to about 5%, or about 0.1% to about 2%, or about 0.3% to about 20%, or about 0.3% to about 18%, or about 0.3% to about 16%, or about 0.3% to about 15%, or about 0.3% to about 12%, or about 0.3% to about 10%, or about 0.3% to about 8%, or about 0.3% to about 6%, or about 0.3% to about 5%, or about 0.3% to about 2%, or about 0.5% to about 20%, or about 0.5% to about 18%, or about 0.5% to about 16%, or about 0.5% to about 15%, or about 0.5% to about 12%, or about 0.5% to about 10%, or about 0.5% to about 8%, or about 0.5% to about 6%, or about 0.5% to about 5%, or about 0.5% to about 2%, or about 1% to about 20%, or about 1% to about 18%, or about 1% to about 16%, or about 1% to about 15%, or about 1% to about 12%, or about 1% to about 10%, or about 1% to about 8%, or about 1% to about 6%, or about 1% to about 5%, or about 3% to about 20%, or about 3% to about 18%, or about 3% to about 16%, or about 3% to about 15%, or about 3% to about 12%, or about 3% to about 10%, or about 3% to about 8%, or about 3% to about 6%, or about 3% to about 5%, or about 4% to about 20%, or about 4% to about 18%, or about 4% to about 16%, or about 4% to about 15%, or about 4% to about 12%, or about 4% to about 10%, or about 4% to about 8%, or about 4% to about 6%, or about 4% to about 5% w/w powder prebiotic.

In a particular embodiment, the composition comprises from about 0.1 to about 30% w/w, or from about 3% to about 8% w/w, or from about 4% to about 6% w/w, or about 5% w/w olive squalene. In a particular embodiment, the composition comprises from about 0.1 to about 30% w/w, or from about 3% to about 8% w/w, or from about 4% to about 6% w/w, or about 5% w/w olive squalene. In a particular embodiment, the composition comprises about 5% w/w flax seed oil. In a particular embodiment, the composition comprises about 1% w/w colloidal oatmeal. In a particular embodiment, the composition comprises from about 0.1 to about 2% w/w, or about 0.2 to about 1.5%, or about 0.2 to about 0.6%, or about 0.3%, or about 0.5 to about 1.3%, or about 0.8 to 1.2%, or about 1% w/w vitamin E.

The inventors have unexpectedly identified that combination of the prebiotics olive squalene and pomegranate seed oil; and the combination of the prebiotics olive squalene and vitamin E are synergistic. Accordingly, in a particular embodiment, the composition comprises from about 0.1% to about 10% w/w olive squalene, about 3% to about 8% w/w, or about 4 to about 7% w/w, or about 5% w/w, or about 0.5% to about 3% w/w, or about 0.5 to about 2% w/w, or about 1% w/w olive squalene, and from about 0.1 to about 10% w/w pomegranate seed oil, or about 2 to about 8% w/w, or about 3 to about 7% w/w, or about 4 to about 6% w/w, or about 3 to about 5% w/w, or about 5% w/w, or about 0.5% to about 3% w/w, or about 0.5 to about 2% w/w, or about 1% w/w pomegranate seed oil. In a particular embodiment, the composition comprises from about 0.1% to about 10% w/w olive squalene, about 3% to about 8% w/w olive squalene, or about 4 to about 7% w/w olive squalene, for example from about 5% w/w olive squalene, and from about 0.1 to about 3% w/w, or about 0.1 to about 2% w/w, or about 0.2 to about 1.5%, or about 0.2 to about 0.6%, or about 0.3%, or about 0.5 to about 1.3%, or about 0.8 to 1.2%, or about 1% w/w vitamin E.

In a particular embodiment, the composition comprises from about 0.1% to about 10% w/w olive squalene, or about 3% to about 8% w/w, or about 4 to about 7% w/w, or about 5% w/w, or about 0.5% to about 3% w/w, or about 0.5 to about 2% w/w, or about 1% w/w olive squalene; and from about 0.1 to about 10% w/w pomegranate seed oil, or about 2 to about 8% w/w, or about 3 to about 7% w/w, or about 4 to about 6% w/w, or about 3 to about 5% w/w, or about 5% w/w, or about 0.5% to about 3% w/w, or about 0.5 to about 2% w/w, or about 1% w/w pomegranate seed oil; and from about 0.1 to about 3% w/w, or about 0.1 to about 2% w/w, or about 0.2 to about 1.5%, or about 0.2 to about 0.6%, or about 0.3%, or about 0.5 to about 1.3%, or about 0.8 to 1.2%, or about 1% w/w vitamin E.

In various embodiments, the fragrance is selected from the group consisting of rose water, orange blossom, rose gardenia, peony, white jasmine, ylang ylang oil, geranium oil, rose oil, and a combination of any two or more thereof. The fragrance may be added as an oil or a powder.

In various embodiments, the antioxidant is selected from the group consisting of vitamin E, resveratrol, squalene, vitamin C, β-carotene, retinyl acetate, retinyl palmitate, retinol, niacinamide, green tea, green tea extract, caffeine, bakuchiol, licorice root, and a combination of any two or more thereof. In various embodiments, the antioxidant is selected from the group consisting of vitamin E, resveratrol, squalene (for example, olive squalene), vitamin C, β-carotene, retinyl acetate, retinyl palmitate, retinol, niacinamide, pomegranate seed oil, flaxseed oil, and a combination of any two or more thereof.

In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids (e.g. lactic acid, glycolic acid), beta-hydroxy acids (e.g. salicylic acid), vitamin B5, seaweed, seaweed extract, silicone, xylitol, oat extract, oatmeal powder, colloidal oatmeal, aloe vera, and a combination of any two or more thereof. In various embodiments, the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids (e.g. lactic acid, glycolic acid), beta-hydroxy acids (e.g. salicylic acid), vitamin B5, seaweed, seaweed extract, silicone, xylitol, oat extract, oatmeal powder, colloidal oatmeal, aloe vera, sunscreen, lactic acid, Camellia oleifera (camellia oil, available e.g. from Go Native (NZ)), hemp seed oil, Hippophae (sea buckthorn) fruit oil, and a combination of any two or more thereof.

In various embodiments, the antimicrobial is selected from the group consisting of zinc, salicylic acid, azelaic acid, benzoyl peroxide, and a combination thereof. In various embodiments, the antimicrobial is selected from the group consisting of zinc, azelaic acid, benzoyl peroxide, tea tree oil, salicylic acid, and a combination thereof.

In various embodiments, the aluminium salt is aluminium chlorohydrate salt, or other salts commonly used in antiperspirants.

In various embodiments, the mineral pigment is zinc oxide and/or titanium dioxide. Mineral pigments are known to protect against UV radiation e.g. from the sun.

In various embodiments, the odour absorbant or neutraliser is zinc ricinoleate or sodium lauryl sarcosinate.

In various embodiments, the sunscreen agent is oxybenzone, avobenzone, octisalate, octocrylene, homosalate, or octinoxate. Mineral sunscreens use zinc oxide and/or titanium dioxide.

A skilled worker will also understand that when the composition is applied in combination with an aqueous phase, the additional additives described may also be provided in the aqueous phase.

### Improving Appearance of Skin or Signs of Aging

The compositions described herein are useful for improving the appearance of skin or improving signs of aging of the skin. Improving the appearance of skin or a sign of aging may include effects that include, but are not limited to, that skin looks more radiant, skin looks healthier, skin feels more hydrated, pores are reduced in size, skin feels softer, skin looks clearer, moisture is increased, sebum production is decreased, and reducing the appearance of wrinkles, dryness, roughness, dullness, spots including age spots, and impurities.

The compositions described herein are also useful for improving the appearance of skin, including dry skin, in infants. Acidity in body fluids in infants can be caused by acidic saliva and changes in the gastrointestinal tract. This can lead to dry skin problems in infants, including teething rash on the mouth, chin, neck or chest from saliva, and nappy rash from stool or urine.

Pore size, skin moisture content, sebum productions, wrinkles, spots, keratin and impurities may all be measured using a Skin analyser device (eg. (Dermo Prime Viso, CHOWIS, South Korea). An Artificial Intelligence powered skin analyser device quantitatively measures changes in various skin parameters such as hydration, sebum, pores, wrinkles, spots/pigmentation, impurities, keratin, skin tone, blackheads, and skin sensitivity. The device is equipped with a moisture sensor and utilises advanced optic technology with interchangeable lenses to measure up to 10 different skin measurements and performs accurate analysis of high-resolution images via the DermoBella app installed in android (Google Inc.) or iOS (Apple) tablets or smartphones. To test for keratin, keratin strips (Chowis, South Korea) can be used in conjunction with the skin analyzer. Keratin strips can be placed sticky side down on the skin with light pressure applied, and left for 10 seconds before removing to analyze using the skin analyzer. The device is used according to the protocols as set out in the Examples herein. Skin parameter measurements are taken at the same skin site each time.

The degree of impurities such as redness can be analysed by measuring the amount of porphyrin. It is indicated as scarlet, orange light in response to a specific wavelength range of UV light.

Impurities are seen as either a scarlet colour or a yellow-green colour. These can be analysed separately, but the device combines the two and classifies them as impurities. The index is computed by a percentage against image size. There are no arbitrary values that classify the degree of redness severity based on the amount of porphyrin detected.

### Form of the Topical Composition

In various embodiments, the composition has a viscosity of from about 2,500,000 to about 8,000,000 cP at 25° C., for example from about 3,000,000 to about 7,500,000, about 3,000,000 to about 7,000,000, about 3,500,000 to about 7,500,000, about 3,500,000 to about 7,000,000, about 3,600,000 to about 7,000,000 about 3,700,000 to about 7,000,000, about 3,800,000 to about 7,000,000, about 3,900,000 to about 7,000,000, about 4,000,000 to about 7,000,000, about 4,000,000 to about 6,900,000, about 4,000,000 to about 6,800,000, about 4,000,000 to about 6,700,000, about 4,000,000 to about 6,600,000, about 4,000,000 to about 6,500,000, about 4,000,000 to about 6,400,000, about 4,000,000 to about 6,300,000, about 4,000,000 to about 6,200,000, about 4,000,000 to about 6,100,000, about 4,000,000 to about 6,000,000, about 4,000,000 to about 5,000,000, about 4,100,000 to about 7,000,000, about 4,200,000 to about 7,000,000 about 4,300,000 to about 7,000,000, about 4,400,000 to about 7,000,000, about 4,500,000 to about 7,000,000, about 4,500,000 to about 6,000,000 about 4,500,000 to about 6,900,000, about 4,500,000 to about 6,800,000, about 4,500,000 to about 6,700,000, about 4,500,000 to about 6,600,000, about 4,500,000 to about 6,500,000 cP at 25° C. In various embodiments, the composition has a viscosity of about 4,500,000, 4,600,000, 4,700,000, 4,800,000, 4,900,000, 5,000,000, 5,100,000, 5,200,000, 5,300,000, 5,400,000, 5,500,000, 5,600,000, 5,700,000, 5,800,000, 5,900,000, 6,000,000, 6,100,000, 6,200,000, 6,300,000, 6,400,000, or 6,500,000 cP at 25° C.

Viscosity may be measured at 25° C. using a Brookfield LVDVI Prime using Brookfield Helipath Spindle (S94 or S95 or S96) set at 0.5 RPM. A skilled worker will appreciate other methods that can be used to measure viscosity and that viscosity measurements may vary depending on the method used.

In various embodiments, the composition has a shelf-life of at least 3 months at 25° C., at 60% RH (relative humidity). In various embodiments, the composition has a shelf-life of at least 6 months, at least 12 months or at least 24 months at 25° C., at 60% RH (relative humidity).

### Method of Preparing Topical Composition

In one aspect, the invention provides a method of manufacturing a topical composition comprising Micrococcus luteus Q24 comprising the steps of:
a) heating a mixture of an oil vehicle and a viscosity modifier to provide a melt mixture,
b) cooling the melt mixture to a temperature in the range of 38° C. to 28° C.,
c) adding Micrococcus luteus Q24 to the melt mixture from step b) to provide a uniform mixture,
d) cooling the mixture from step c) to room temperature to provide the composition.

In some embodiments, a dispersing agent is added during step (a) or before step (b).

Micrococcus luteus Q24 has been found to be heat-sensitive at elevated temperatures (>40° C.). Heating results in loss of cell viability. The viscosity modifiers used in the invention are solid or semisolid at room temperature and are usually processed at elevated temperature. These solid viscosity modifiers pose unique challenges in the preparation of compositions comprising live and viable microorganisms such as Micrococcus luteus Q24. The applicants have after considerable trial and effort developed methods of preparing the compositions that allow efficient manufacturing of the composition while maintaining optimal viability of the probiotic.

In various embodiments, the melt mixture is cooled to a temperature in the range of about 35 to about 28° C., for example about 35 to about 29, about 35 to about 30, about 35 to about 31, about 34 to about 28, about 34 to about 29, about 34 to about 30, about 33 to about 30, about 32 to about 29, about 31 to about 29° C. In various embodiments, the melt mixture is cooled to a temperature of about 35, 34, 33, 32, 31, 30, 29 or 28° C. In some embodiments, cooling can be achieved by simply removing the melt mixture from a heating source such as a hot plate used for heating step (a). In some embodiments, the melt mixture can be stirred while cooling, for example by placing on a magnetic stirrer plate, and Micrococcus luteus Q24 can be added once the melt mixture has cooled to the required temperature for step (b).

Embodiments where Micrococcus luteus Q24 raw ingredient is added to the formulation at elevated temperature can require the maintenance of the formulation at that temperature, which can limit the ability to upscale the process to make larger batches. To address this issue the applicants have worked to achieve balances between cooling and timing of addition of Micrococcus luteus.

In one aspect, the invention provides a method of manufacturing a topical composition comprising Micrococcus luteus Q24 comprising the steps of:
a) heating a mixture of an oil vehicle and a viscosity modifier to provide a melt mixture,
b) cooling the melt mixture to a temperature between about 22° C. to about 27° C.,
c) adding Micrococcus luteus Q24 to the cooled mixture from step b), and
d) mixing to provide a uniform mixture.

In various embodiments, the mixing in step (d) may be achieved using a high shear homogenizer or an overhead stirrer. In various embodiments, the melt mixture is cooled to room temperature in step (b), and the method thus advantageously does not comprise a step of heating the Micrococcus luteus Q24 above room temperature, thereby enhancing the microbiological stability of the live probiotic. An additional advantage is that there is no need to manage addition of Micrococcus luteus Q24 raw ingredient during the cooling stage, providing advantages for larger-scale batch production.

The topical composition described herein may thus be prepared by first mixing an oil vehicle and a viscosity modifier, adding Micrococcus luteus Q24 to the mixture, and homogenising the mixture e.g. for about 1 to 3 minutes, to provide the composition. In various embodiments, the mixture is homogenized with a high shear homogenizer or an overhead stirrer.

The Micrococcus luteus Q24 may be milled or sieved to a particle size of less than 250 μm prior to adding to the mixture. In various embodiments, the particle size of Micrococcus luteus Q24 is less than about 250 μm, or less than about 100 μm. Suitable milling and sieving techniques will be apparent to a skilled worker. For example, the Micrococcus luteus Q24 may be milled using a dry powder comill (e.g. Quadro Powder Mills, such as U10 or the U21). Smaller particle sizes may be advantageous for providing improved spreadability of the composition, enhancing dispersibility when combined with the aqueous phase, and providing improved sensory properties of the composition i.e. is not grainy or gritty.

In various embodiments, Micrococcus luteus Q24 have a particle size (Dv90) of less than about 300 μm, or less than about 250 μm. Dv90 may be measured by the laser diffraction method for particle size analysis of dry powder dispersion (Malvern Instruments, USA). A skilled worker will appreciate that particles with a Dv90 of less than 300 μm may entirely pass through a 250 μm sieve.

The stability of the balm formulations of the invention can be investigated using standard tests for stability with respect to time, temperature, humidity and physical forces (syneresis). Syneresis is separation of liquid phase from solid phase of a balm/gel-based system due to shrinkage of the matrix over time. This phenomenon is a slow process under real time storage normal conditions. Syneresis is undesirable in consumer products such as balm formulations. The physical appearance as well as uniformity of dose and sensory properties can be significantly affected due to syneresis. Wax systems tend to have high physical stability because of their strong wax-crystal bond interactions causing lattice formation (Ivanovszky, L. Waxes: “Colloidal properties and systems”, Journal of Polymer Science, 58(166), 273-288, 1962). With the addition of further ingredients, such as oils and butters, the lattice structure is interrupted, which alters the waxes resilience to external forces. Syneresis occurs when the formulation structure is broken, resulting in the leakage of oil.

A small propensity for syneresis (for example a composition exhibiting less than 15% syneresis at 13,000 rpm) improves sensory properties of a composition for skin, as it enables easier spreadability of the composition and absorption onto the skin. It can also be useful for analytical testing as it allows for easier sampling and mixing of the product. However, a high propensity for syneresis (e.g. more than 20% syneresis at 13,000 rpm) can alter the product properties over time. This inconsistency and lack of physical stability is not ideal in a skin-care formulation in market, and moreover is likely to impact on the stability of a live culture present in the formulation.

### Methods of Use

The invention also relates to a method of improving the appearance of skin or at least one sign of aging in a subject in need thereof. The method comprises applying to the skin of a subject in need thereof a topical composition of the invention. The compositions of the invention may be used as a balm. The composition may be applied in an amount appropriate to address the skin issue of the subject. A skilled worker will appreciate the composition may be applied to a subject of any age from the very young, including infants, to the very old. The amount applied may vary according to the subject's age, sex, and issue being addressed. A typical application regime may comprise application of the composition of the invention monthly, weekly, daily, or between one and four times daily.

The composition may be applied by hand or with an applicator from a pottle or other container. The amount applied can be selected by the user according to the area to be treated. The composition may be to an area of concern (e.g. dry spots such as elbows, knees etc), or may be applied more broadly to any part of the body.

The compositions of the invention may be used in the same way as a conventional skin balm, and in any daily skin-care regime. A typical regime may comprise use of the composition of the invention once or twice daily. Use of the composition for extended periods, or for time-limited periods e.g. for one to two months is contemplated.

### EXAMPLES

### Materials

Equipment: Unless otherwise stated, ingredients of the balm formulations were mixed using a magnetic stirrer and hot plate (DIAB MS-H280-Pro). Samples for the recovery analysis were mixed using a stomacher (Masticator Basic IUL Instruments, NZ). Cultured colonies were counted using a Q Count Automated Colony Counter (Hunting Tree Bioscience Supplies, New Zealand).

Chemicals: Medium chain triglyceride (Caprylic/Capric Triglyceride) e.g. Miglyol 812 (Ph Eur grade) or Radia 7104 were purchased from Sasol, Hamburg, Germany or Oleon, Malaysia respectively. Tween 80 (Ph Eur grade) was purchased from Sigma (New Zealand). Hydrophobic silica (Aerosil R 972 (Pharma grade) was purchased from Evonik, Germany (supplied by Chemiplas, Auckland, New Zealand). Beeswax, cetostearyl alcohol, cocoa butter, shea butter, pomegranate seed oil, vitamin E, olive squalene, colloidal oatmeal USP were purchased from Pure Ingredients Ltd (New Zealand). Candelilla wax was purchased from Go Native (New Zealand). Emulsifying wax was purchased from Lotus Oils Ltd (New Zealand). Self-emulsifying glyceryl monostearate (Imwitor® 960K) was purchased from IOI Oleo (Germany). Ethyl cellulose was purchased from Colorcon (UK). Olive oil was purchased from Lipoid (Germany). Distilled water was used for sample and culture preparations. All other chemicals and solvents were of analytical grade (Sigma, New Zealand).

Active probiotic Micrococcus luteus Q24 freeze dried raw ingredient powder was produced by Blis Technologies. Recovery analysis and dilutions were prepared using analytical grade Phosphate Buffered Saline (PBS) solution (Oxoid, Dulbecco A), purchased from Thermofisher. The sample dispersions were prepared using non-filter stomacher bags (BagLight PolySilk Transparent, 400 mL) and filter stomacher bags (BagPage+Full-page filter bag with Microperforated filter, 400 mL), both purchased from Interscience, New Zealand. Bacteria cultures were grown on CABK12, sheep and human blood agar plates, purchased from Fort Richard Laboratories, New Zealand. De-ionised water was used for preparing buffers and agar medium.

Alternatively to purchasing CAB K12 Agar (Columbia blood agar) plates, they can be prepared using the following ingredients: Columbia blood agar base (bd.com, USA) 44.0 g, Yeast extract (Bacto, bd.com, USA) 5.0 g, Glucose 2.5 g, calcium carbonate (Sigma-Aldrich, USA) 1.0 g; or other amounts of these ingredients in an equivalent weight ratio. A suitable procedure is: suspend CAB K12 agar in 1 L of distilled water in an appropriately sized conical flask or beaker (sufficiently large to not boil over). Cover the flask or beaker with aluminium foil and heat with stirring to boiling on a hot plate. Allow to boil for 1 min to completely dissolve the ingredients, then autoclave the solution at 121° C. for 15 min. Cool to 50° C., mix well for calcium carbonate to evenly suspend, adjust pH if required to 7.3±0.2, and aseptically pour into petri-dishes.

Minimum Inhibitory Concentration (MIC) of the formulation ingredients against Micrococcus luteus Q24 was tested by spreading Micrococcus luteus Q24 freeze dried raw ingredient suspension onto CABK12 agar plates which was prepared by adding 3-5 Micrococcus luteus Q24 colonies (from overnight culture grown on sheep blood agar plate) to 3 mL of PBS. A 1:10 dilution of the ingredient in an oil vehicle was serially diluted to make 5%, 1.25%. 0.625%, 0.156% v/v concentrations. Finally, 20 μL of each concentration was spotted (100%, 10%, 5%, 1.25%. 0.625% and 0.156%) onto segments of the agar plate. The plates were incubated for 24 hours Under 37° C. 5% CO₂. Following incubation, presence of zones of inhibition were recorded to determine MICs. Test results confirmed the ingredients used in the balm formulations shown in Table 1 have no inhibitory activity against Micrococcus luteus Q24.

### Examples 1-4—Preparation of Balm Formulations

Balm Formulations were prepared having the ingredients shown in Table 1. Once the balm formulations were prepared, they were stored at room temperature or refrigerated conditions for at least three days before physicochemical or sensory evaluation.

TABLE 1
Example formulations

| | | Ingredients (w %/w) | | | | | | | |
|---|---|---|---|---|---|---|---|---|---|
| | | | | | | Self- | | | |
| | | | | | | emulsifying | | | |
| Formulation | Formulation | Micrococcus | polysorbate | | Cetostearyl | Glyceryl | Candelilla | Ethyl | Emulsifying |
| Platform | Number | luteus Q24 | 80 | Beeswax | Alcohol | monostearate | wax | Cellulose | wax |
| Bees | BLT-BB1 | 1 | | 3 | | | | | |
| wax | BLT-BB3/a | 1 | | 15 | | | | | |
| | BLT-BB4 | 1 | | 30 | | | | | |
| | BLT-BB5 | 1 | | 40 | | | | | |
| | BLT-BB6 | 1 | | 40 | | | | | |
| | BLT-BB7 | 1 | | 20 | | | | | |
| | BLT-BB9 | 1 | 1 | 20 | | | | | |
| | BLT-BB9B | 1 | 1 | 20 | | | | | |
| Cetostearyl | BLT-BB2 | 1 | | | 12.5 | | | | |
| Alcohol | BLT-BB12 | 1 | 1 | 20 | 5 | | | | |
| | BLT-BB15 | 1 | 1 | 25 | 5 | | | | |
| | BLT-BB19 | 1 | 1 | 20 | 7 | | | | |
| | BLT-BB24 | 1 | 1 | 25 | 5 | | | | |
| | BLT-BB25 | 1 | 1 | 25 | 5 | | | | |
| | BLT-BB26 | 1 | 1 | 25 | 5 | | | | |
| Imwitor | BLT-BB11 | 1 | 1 | | | 15 | | | |
| wax | BLT-BB14 | 1 | 1 | | | 15 | | | |
| | BLT-BB17 | 1 | 1 | | | 5 | | | |
| | BLT-BB23 | 1 | 1 | | | 15 | | | |
| Candelilla | BLT-BB10 | 1 | 1 | | | | 10 | | |
| wax | BLT-BB13 | 1 | 1 | | | | 15 | | |
| | BLT-BB16 | 1 | 1 | 20 | | | 10 | | |
| | BLT-BB18 | 1 | 1 | | | | 10 | | |
| Silica | BLT-BB20 | 1 | 1 | 20 | | | | | |
| | BLT-BB28 | 1 | 1 | 5 | | | | | |
| | BLT-BB29 | 1 | 1 | | | | | | |
| Other | BLT-BB21 | 1 | 1 | 20 | | | | 2 | |
| | BLT-BB22 | 1 | 1 | 20 | | | | | 2 |
| | BLT-BB27 | 1 | 1 | | | | | | |

| | | Ingredients (w %/w) | | | | | | | | | |
|---|---|---|---|---|---|---|---|---|---|---|---|
| Formulation | Formulation | Hydrophobic | Shea | Cocoa | Olive | MCT | Calendula | Pomegranate | | Olive | Vitamin |
| Platform | Number | Silica | Butter | Butter | Oil | Oil | Oil | Oil | Oatmeal | Squalene | E |
| Bees | BLT-BB1 | | 10 | 10 | 77 | | | | | | |
| wax | BLT-BB3/a | | 10 | 10 | 64 | | | | | | |
| | BLT-BB4 | | 20 | 20 | 29 | | | | | | |
| | BLT-BB5 | | 29 | 30 | | | | | | | |
| | BLT-BB6 | | 24 | 25 | 10 | | | | | | |
| | BLT-BB7 | | 20 | 20 | 39 | | | | | | |
| | BLT-BB9 | | 20 | 20 | 38 | | | | | | |
| | BLT-BB9B | | 20 | 20 | 38 | | | | | | |
| Cetostearyl | BLT-BB2 | | | | 76.5 | | 10 | | | | |
| Alcohol | BLT-BB12 | | | 10 | 63 | | | | | | |
| | BLT-BB15 | | | 20 | 48 | | | | | | |
| | BLT-BB19 | | | 30 | 41 | | | | | | |
| | BLT-BB24 | | | 20 | | 48 | | | | | |
| | BLT-BB25 | | | 20 | | 40 | | 5 | 3 | | |
| | BLT-BB26 | | | 20 | | 42 | | | | 5 | 1 |
| Imwitor | BLT-BB11 | | 20 | 20 | 43 | | | | | | |
| wax | BLT-BB14 | | 25 | 25 | 33 | | | | | | |
| | BLT-BB17 | | 25 | 25 | 43 | | | | | | |
| | BLT-BB23 | | 25 | 25 | | 33 | | | | | |
| Candelilla | BLT-BB10 | | 20 | 20 | 48 | | | | | | |
| wax | BLT-BB13 | | 25 | 25 | 33 | | | | | | |
| | BLT-BB16 | | 25 | 25 | 18 | | | | | | |
| | BLT-BB18 | | 20 | 20 | 48 | | | | | | |
| Silica | BLT-BB20 | 2 | 20 | 20 | 36 | | | | | | |
| | BLT-BB28 | 10 | 20 | 20 | 43^{B} | | | | | | |
| | BLT-BB29 | 10 | 25 | 25 | 38 | | | | | | |
| Other | BLT-BB21 | | 20 | 20 | 36 | | | | | | |
| | BLT-BB22 | | 20 | 20 | 36 | | | | | | |
| | BLT-BB27 | | 25 | 25 | | 48 | | | | | |

### Example 1: Wax-Based Balm Formulations

Formulations were prepared having the waxes shown in Table 2 and other ingredients as shown in Table 1.

TABLE 2
Melting points of waxes used in balm formulations

| Wax | Melting point | | Solidifying Process |
|---|---|---|---|
| Beeswax | 62-65° | C. | Rapid |
| Cetostearyl alcohol | 50° | C. | Rapid |
| Candelilla wax | 68.5-72.5° | C. | Medium |
| Self-emulsifying glyceryl | 66-77° | C. | Slow |
| monostearate | | | |
| Emulsifying wax | 45-53° | C. | Slow/Medium |

In a 100 mL glass beaker placed inside another beaker filled with water to create a jacketed water bath, wax was heated on a magnetic stirrer hotplate until a temperature of respective wax (Table 2) was reached (45° C.-70° C.). The temperature of the formulation was measured using an infrared temperature gun or a temperature probe. Once the wax had completely melted (process takes ˜10 min), other ingredients as shown in Table 1 were added slowly to the melted wax with continuous stirring (600-700 rpm) using a magnetic stirrer bar. Once the mixture was completely dissolved (clear solution, ˜10 min), heating was turned off and the beaker moved to another stirrer plate (non-heated). The stirring continued to allow the mixture to cool down slowly.

Once the temperature reached 30° C., Micrococcus luteus Q24 powder was added to the homogeneous mixture slowly. The mixing was achieved using the magnetic stirrer or manual mixing using glass rod to ensure homogeneity.

### Example 2: Wax-Based Balm Formulation; Cold Method

Live Micrococcus luteus Q24 raw ingredient is temperature-sensitive. The inventors therefore sought an alternative approach to the melt method of Example 1. In this alternate method Micrococcus luteus Q24 was added once the formulation had cooled to room temperature. Due to the viscosity at this temperature, a high-shear homogenizer was used in this example.

The formulation BLT-BB9 was made according to Example 1 above, using the ingredients with the proportions listed in Table 1. A corresponding formulation BLT-BB9B (Table 1) was made by varying the method of Example 1 to add Micrococcus luteus Q24 powder after the formulation had cooled to room temperature, rather than once the temperature reached 30° C. The formulation was then mixed using a high shear homogenizer (DLAB D-160 homogeniser, Ultra-Turrax (IKA Instruments, Germany, via Lab Supply, New Zealand) for a fixed period of time to avoid the addition of heat due to friction. The homogenizer was operated for 3-5 minutes with gradual speed up from about 8,000 rpm (speed 1 on the instrument dial) up to about 18,000 rpm (speed 4) then down to about 8,000 rpm).

Cell counts of formulations BB9 and BB9B were taken at TO, and at T2 weeks to compare with their relative counts. The sensory properties of these balms prepared using the different techniques were also compared.

A statistically significant difference in cell counts between the two samples was not observed. Based on these preliminary results, it can be concluded that the use of the homogenizer does not impact the cell count of the product. An informal sensory assessment suggested that there was little difference in the application sensory properties, however visual appearance of formulation BLT-BB9B of Example 2 was preferred over formulation BLT-BB9 of Example 1.

### Example 3: Ethyl Cellulose-Based Balm Formulations

The following example was used to prepare formulation BLT-BB21 as shown in Table 1. In a 100 mL glass beaker, oil was heated on a magnetic stirrer hotplate until a temperature of 160-170° C. was reached (measured using an infrared temperature gun or a temperature probe). Ethyl cellulose was added slowly to the heated oil with continuous stirring (600-700 rpm) using magnetic stirrer bar. The stirring speed used to achieve a good solubilization of the ethyl cellulose are between 600 and 700 rpm, higher speeds were found to form bubbles. Slow addition of the ethyl cellulose to the oil and continuous stirring were used to avoid formation of lumps of ethyl cellulose. Once the ethyl cellulose was completely dissolved (clear solution, ˜40 min), heating was turned off and the beaker moved to another stirrer plate (nonheated). The stirring was continued to allow the ethyl cellulose-oil mixture to cool down slowly. Once the temperature reached 60-70° C., polysorbate 80 was added followed by wax with continuous stirring until a homogeneous mixture was achieved. Once the temperature reached 30° C., Micrococcus luteus Q24 powder was added to the mixture slowly.

### Example 4: Balm Formulations Containing Silica

Balm formulations containing silica were produced following a similar methodology as the balm formulations for Examples 1 and 3, with the silica being added once the formulation had cooled to room temperature. As for Example 2, a high shear homogenizer was used for 3-5 minutes with gradual speed up from 1-4 then down from 4-1 on the instrument speed dial, to blend the silica with the cooled formulation.

Micrococcus luteus Q24 was used for all microbiological testing samples and pilot efficacy trial.

### Example 5: Stability Testing—Temporal

The viability of Micrococcus luteus Q24 in balm formulations was assessed under different storage conditions temperature and humidity according to ICH guidelines for stability testing (ICH Q1AR2). A 150 g batch of each of the six balm formulations BLT-BB14, BLT-BB15, BLT-BB23, BLT-BB24, BLT-BB25, and BLT-BB26 was prepared in a glass beaker (250 mL) following procedure as described in Example 1.

TABLE 3
Formulations used within the Stability Experiment,
their relative platforms (base ingredients)

| Formulation | |
|---|---|
| Name | Platform and Key Ingredients |
| BLT-BB14 | Self-emulsifying glyceryl monostearate (Olive Oil) |
| BLT-BB15 | Beeswax and Cetostearyl Alcohol (Olive Oil) |
| BLT-BB23 | Self-emulsifying glyceryl monostearate (MCT Oil) |
| BLT-BB24 | Beeswax and Cetostearyl Alcohol (MCT Oil) |
| BLT-BB25 | Beeswax and Cetostearyl Alcohol (MCT Oil) + |
| | Functional actives (Pomegranate seed oil and oatmeal) |
| BLT-BB26 | Beeswax and Cetostearyl Alcohol (MCT Oil) + |
| | Functional actives (Olive squalene and Vitamin E) |

The ingredients of these formulations are described in Table 1.

Balm formulations were stored in 30 mL glass pottles, with a thin plastic seal and plastic twist-top lid. 15 g of each balm formulation was filled in the glass pottles and stored under Real time (25° C.±2° C./60% RH). The results are shown in FIG. 2.

For comparison, a deodorant stick formulation was made following the process described in WO2006/104403 Example 2.

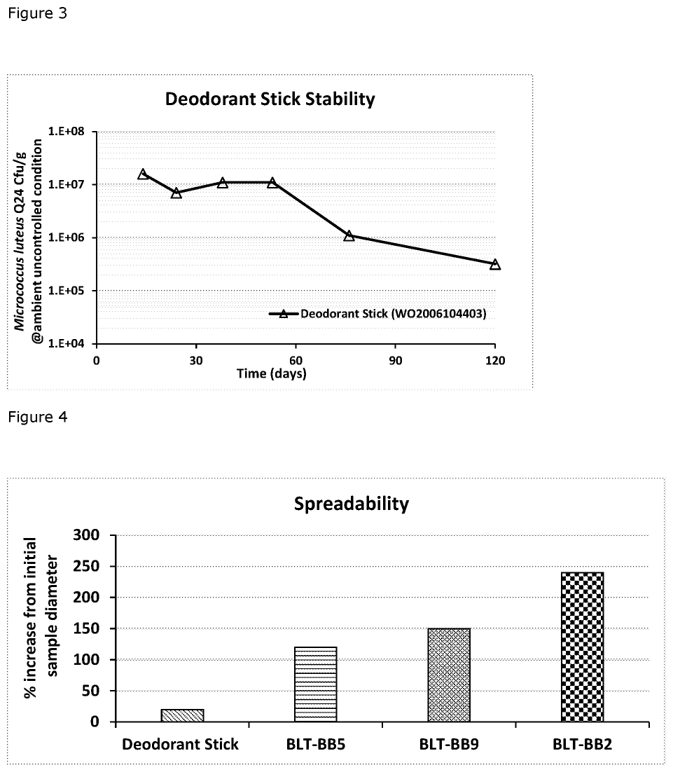

FIG. 3 shows microbiological cell counts of the deodorant stick disclosed in WO2006/104403 under controlled storage conditions (25° C./60% RH). The hard deodorant stick formulation of WO2006/104403 contains 92% w/w shea butter/cocoa butter, and has a very high viscosity of greater than 9,375,000 cP and therefore low spreadability, thus it cannot be applied to the skin as a balm. The deodorant stick formulations of WO2006/104403 are formed at a high melt temperature (>40° C.) at which addition of Micrococcus luteus Q24 is detrimental for storage over long duration. The high heat results in weaker cells which are viable for initial stability points (1 month) and then decline rapidly, indicating they are not shelf stable. A drop in cell counts is observed within 1 month, compared to balm formulations of the invention that maintain viability for at least three months.

### Example 6: Viscosity and Spreadability

### Viscosity

Viscosity of a number of different formulations was measured using Brookfield LVDV1-Prime using Brookfield Helipath Spindles (S94, S95 or S96). The results are shown in Table 4.

TABLE 4
Average Viscosity (cP) of formulations at 25° C. ± 2° C.

| | | | | Capric/ | | | |
|---|---|---|---|---|---|---|---|
| | | Hydrophobic | | caprylic | | Aqueous | |
| | | silica | Tween | triglyceride | Olive | Cream BP | |
| | Blis Q24 | (AR972) | 80 | (MCT) | oil | (SLS free) | Viscosity |
| Formulation | (wt %) | (wt %) | (wt %) | (wt %) | (wt %) | (wt %) | (cP) |
| Aqueous | 1% | — | — | — | — | 99% | 155,000^{a} |
| cream | | | | | | | |
| Serum | 1% | 4% | 1% | 94% | — | — | 144,000^{b} |
| Serum | 1% | 5% | 1% | 93% | — | — | 439,000^{c} |
| Serum | 1% | 6% | 1% | 92% | — | — | 645,000^{d} |
| Serum | 1% | 7% | 1% | 91% | | | 1,410,000^{e} |
| Balm BLT- | 1% | Formulated with ingredients as per Table 1 above | | | | | 4,615,000^{f} |
| BB15 | | | | | | | |
| Balm BLT- | 1% | Formulated with ingredients as per Table 1 above | | | | | 4,871,300^{g} |
| BB25 | | | | | | | |
| Deodorant | 0.1% | Shea butter 52.7%; cocoa butter 39.6%; | | | | | >9,375,000^{g} |
| stick - | | potassium alum 7.6% | | | | | |
| WO | | | | | | | |
| 2006/104403 | | | | | | | |

Spindle and speed
^{a}S95, 1 rpm
^{b}S94, 0.5 rpm
^{c}S95, 0.5 rpm
^{d}S96, 0.5 rpm
^{e}S95, 0.5 rpm
^{f}S96, 0.1 rpm
^{g}S96, 0.1 rpm.

The serum formulations were measured to have an average viscosity of 144,000-1,410,000 cP, with viscosity increasing with silica content (Table 6). The formulations BB15 and BB25 were measured to have an average viscosity of U.S. Pat. Nos. 4,615,000 and 4,871,300 cP respectively. The viscosity of the deodorant stick formulation of Example 2 of WO 2006/104403 is very high, and was outside the measurement range of the equipment used (maximum measurable viscosity of 9,375,000 cP) (Table 4).

### Comparative Observations and Testing-Viscosity; Microbiological Stability

A grape seed oil suspension was made following the process described in WO2006/104403: Example 3, using Micrococcus luteus Q24 raw ingredient with a particle size (Dv90) of 290 μm. This formulation was thin and runny with non-homogenous raw ingredient distribution, gritty due to non-homogenous distribution of particles and not suitable for use as a balm based on visual thickness (too runny).

A deodorant stick formulation was made following the process described in WO2006/104403: Example 2, using Micrococcus luteus Q24 raw ingredient with a Dv90 of 290 μm. Formulation 2 was thick, almost solid with homogenous raw ingredient distribution; solid to start, non gritty once melted, and not suitable for use as a balm based on visual thickness (too thick).

A Cetomacrogol cream formulation was made according to Australian New Zealand Clinical Trials Registry (ANZCTR) trial ACTRN12616000022460 by mixing Q24 raw ingredient (250 μm sieved) killed by gamma irradiation into commercially purchased Cetomacrogol cream (HealthE Non-ionic cream, Jaychem, New Zealand). Formulation 3 was of medium thickness that settled when dispensed with homogenous raw ingredient distribution, non-gritty and suitable for facial skin application based on visual thickness. Micrococcus luteus Q24 was found to be highly unstable in Formulation 3. A 7-log drop was observed after just 7 days and no viable cell count was found at 14 days when stored at 25° C./60% RH.

### Comparative Testing—Microbiological Stability

A formulation was prepared by weighing 19.8 g of Cetomacrogol 100BP cream (HealthE, Non-ionic cream, B62236, Jaychem, New Zealand) into a stomacher bag using a sterile spoon. 0.2 g (2% in final formulation) of Micrococcus luteus Q24 raw ingredient was added to the Cetomacrogol cream, sealed and hand mixed until a homogeneous cream was formed. The cream was then placed in a stomacher and mixed for an additional 5 minutes. The Cetomacrogol cream containing Micrococcus luteus Q24 was then dispensed into 30 ml glass vials, enumerated in triplicate. In a microcentrifuge tube (Eppendorf, USA), 0.1 g of Micrococcus luteus Q24 serum or cream was weighed and diluted with 0.9 g of warm (37° C.) Phosphate buffered saline and Polysorbate 80 (0.1% w/w). The mixture was homogenised by shaking using a vortex mixer (auto vortex) at 2800 rpm for 5 min under ambient conditions (20° C.±2° C.) to obtain a homogeneous dispersion. 100 μL was then appropriately serially diluted with PBS, spread plated onto hBaCa agar plate and incubated at 37° C., 5% CO₂ for 28 h-48 h. The colonies were counted and calculated for bacteria concentrations in CFU/g, using a Q-Count Automatic Colony Counter (Spiral Biotech, New Zealand). The vials were then placed into an incubator set at 25° C./60% RH for stability testing at time points 7, and 14 days. Micrococcus luteus was found to be highly unstable in this formulation. A 7-log drop was observed after just 7 days and no viable cell count was found at 14 days when stored at 25° C./60% RH.

### Spreadability

The spreadability of the balm formulations (Table 5) was measured using a procedure similar to that described by Saleh et al., “Evaluation of Skin Permeation and Analgesic Activity Effects of Carbopol Lornoxicam Topical Gels Containing Penetration Enhancer” The Scientific World Journal, (2014). A 1 cm diameter circle was drawn onto a glass slide and balm was spread evenly within this 1 cm diameter. This was covered with a second glass slide to which was then added a weight of 200 g, with caution to maintain a parallel lowering angle. The weight was left on the slide for 30 seconds, then lifted and the change in diameter measured. Results are shown in FIG. 4.

TABLE 5
Formulations used in spreadability test, ingredients and properties

| Formulation | | |
|---|---|---|
| Name | Ingredients Used | Properties |
| BLT-BB2 | Cetostearyl alcohol-based | Very soft formulation with minimal structural |
| | formulation with olive and | integrity. |
| | calendula oil. | |
| BLT-BB9 | Beeswax-based formulation with | Thick balm-like formulation with good |
| | olive oil. | spreadability. |
| BLT-BB5 | Beeswax-based formulation with | Formulation is very difficult to apply with hard |
| | no oil component, only butters. | consistency. |

The property of spreadability is important for application of the balm to the skin. These results show the spreadability of balms in the order BLT-BB2 > BLT-BB9 > BLT-BB5 (FIG. 4). BLT-BB2 had no beeswax component (only cetostearyl alcohol), and BLT-BB5 had a high proportion of beeswax present.

### Example 7: Physical Stability

A centrifugal acceleration syneresis test was performed to compare syneresis propensity of the balm formulations BLT-BB143, BLT-BB15, BLT-BB23, BLT-BB24, BLT-BB25, and BLT-BB26, and that of a deodorant stick formulation made following the process described in WO2006/104403 Example 2, after 20 minutes of centrifugal acceleration at speeds of 1000, 1500, 5000 and 13,000 rpm. 1 g of each balm formulation was weighed in a separate 1.5 mL Eppendorf tube and centrifuged at 13,000 rpm for 20 minutes. The volume of liquid leaked was transferred into another Eppendorf tube and weighed. This method was repeated for the other centrifuge speeds (1500, 5000 and 13000 rpm).

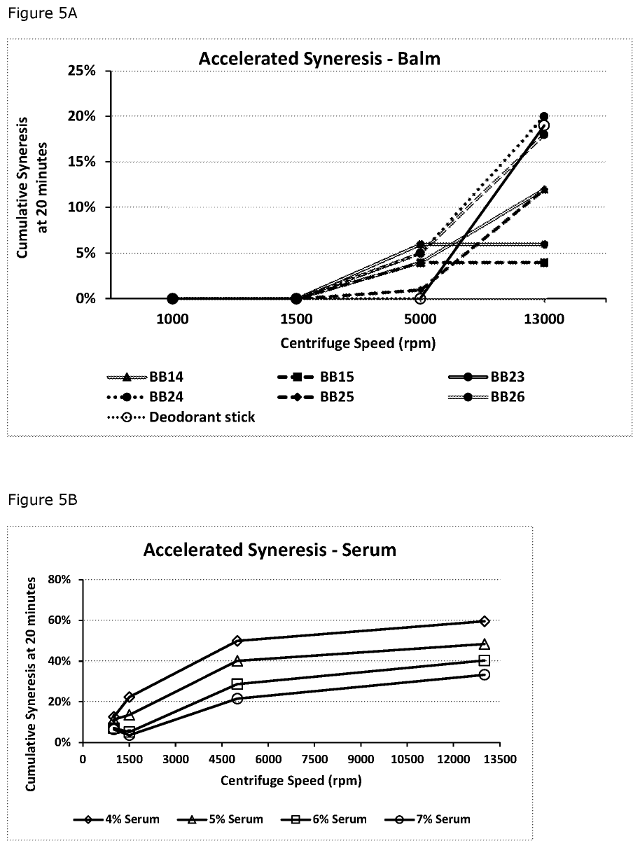

At 13000 rpm, glyceryl monostearate-based formulations (BLT-BB14 and BLT-BB23) showed low cumulative syneresis of only 11.6% and 5.8% after 20 minutes centrifugal acceleration, while the beeswax and cetostearyl alcohol-based formulations BLT-BB24 and BLT-BB26 display lower physical stability with having 20% and 17.9% cumulative syneresis after 20 minutes of centrifugal acceleration (FIG. 5A). This suggests that BLT-BB15 is the most physically stable formulation having only 4.5% cumulative syneresis after 20 minutes. All balm formulations tested showed syneresis of less than 20% at 13000 rpm. Therefore, the balms will likely remain intact when exposed to ‘real-world’ forces.

Unlike the balm formulations, the hard deodorant stick formulation of Example 2 of WO2006/104403 showed no syneresis at all up to 5,000 rpm. At 13,000 rpm, it displayed a higher cumulative syneresis of about 19%.

Unlike the balm formulations, the serum formulations shown in Table 4 all showed syneresis of greater than 20% at 5,000 rpm (FIG. 5B). At 13,000 rpm, the serum formulations displayed a higher cumulative syneresis of between 30 and 60%.

It is interesting to note that while the formulations BLT-BB15 and BLT-BB24 only differ in the use of oil vehicle, BLT-BB15 containing olive oil is more stable with respect to syneresis than formulation BLT-BB24 which contains MCT oil.

FIG. 5A shows syneresis measurements for slower centrifuge speeds of 1000, 1500 and 5000 rpm. As shown in the Figure, for all formulations at the 20-minute time point there is an increase in syneresis as the centrifuge speed (rpm) increases. It is also clear that at lower speeds (1000 and 1500 rpm) there is less syneresis occurring. This supports the conclusion that the balm formulations will likely have good physical stability when exposed to ‘real-world’ shear forces.

### Example 8: Safety and Efficacy—EpiDerm Model

The safety and efficacy of Micrococcus luteus Q24 bacterial cells was evaluated using the EpiDerm 3D Skin Model.

### Methodology

### EpiDerm Human 3D Skin Model Culture (Skin Model)

Upon receipt, the final development of the immature EpiDerm (EPI-201-4D; MatTekCorporation, Ashland, MA, USA) tissues was completed following the manufacturer's instructions. In brief, the tissues were transferred from the agarose-containing gel on which they were shipped to cell culture plates containing pre-warmed differentiation medium (EPI-201-DM). They were then maintained at 37° C. in a 5% CO₂ atmosphere for 4 days, with 2 or3 changes to fresh differentiation medium during that period. Finally, the fully developed EpiDerm tissues were transferred to fresh plates containing standard culture medium (EPI-100-NMM) for use in each experiment.

### Dose Response Experiment for Tolerability

The maturation of 24 immature EpiDerm tissues was performed as described above. The fully differentiated tissues were randomly assigned to receive either PBS (negative control) or Micrococcus luteus Q24 at one of 5 dose levels (1×10⁵ cfu/ml to 1×10⁹ cfu/ml), with 4 replicate tissues used per treatment. The apical surfaces of the tissues were treated with 30 μL of the treatment solutions, and the tissues were then cultured for 24 h at 37° C. in a 5% CO₂ atmosphere. At the end of that period the tissues were gently rinsed with PBS and their viabilities were assessed using the MTT viability assay (Mosmann, T. (1983). Journal of Immunological Methods, 65 (1-2), 55-63, Kubilus, J. (1996). In Vitro and Molecular Toxicology, 9 (2), 157-166.).

### Safety and Efficacy Experiment

The maturation of 36 immature EpiDerm tissues was performed as described above. The fully differentiated tissues were numbered from 1-36 and were randomly assigned to receive one of six combinations of one of three main treatments (PBS, Micrococcus luteus Q24 or vitamin C) with either PBS as negative control or 0.5% sodium dodecyl sulphate (SDS) exposure treatment (+0.5% SDS) randomly represented on each of five 6-well cell culture plates. The remaining 6 tissues, which were destined for histology or else to receive 5% SDS, were grouped together on the sixth cell culture plate. Following maturation, the 36 tissues were cultured in the assay medium for 24 h at 37° C. in a 5% CO₂ atmosphere, after which the baseline (day 0) conditioned media samples were collected and stored frozen at −80° C. for cytokine measurement. For 4 days starting from day 0, each tissue was treated daily with the appropriate main treatment solution (PBS, Micrococcus luteus Q24 or vitamin C) before the tissues were returned to the incubator maintained at 37° C. with a 5% CO₂ atmosphere. Tissues that were treated with Micrococcus luteus Q24 or the PBS vehicle had 30 μL of the appropriate solution applied to the apical surfaces of the tissues, after the remnants of the previous day's doses were rinsed off with PBS. Vitamin C (50 μg/mL) was instead delivered in the culture medium. On day 4 conditioned media samples were collected and stored frozen at −80° C. for cytokine measurement. 33 of the tissues were then exposed for 30 μL of 0.5% or 5% SDS in PBS, or to PBS as negative control. After 1 h these tissues were rinsed with PBS and dabbed dry with cotton buds before re-application of the appropriate main treatment (PBS, Micrococcus luteus Q24 or vitamin C) and transferred to plates containing fresh medium. The remaining 3 tissues (destined for histology) were simply fed fresh medium and given the appropriate main treatment. All tissues were returned to the incubator for a further 24 h at 37° C. in a 5% CO₂ atmosphere, after which time the day 5 conditioned media were collected and stored frozen at −80° C. All tissues were gently rinsed with PBS and the 3 histology tissues were fixed in neutral buffered formalin. The viabilities of the other 33 tissues were assessed using the MTT viability assay.

### MTT Viability Assay (Safety)

EpiDerm tissues on cell culture inserts were added to 6-well cell culture plates containing 0.9 mL of 1 mg/mL MTT in DMEM medium (Thermo Fisher Scientific) and were cultured for 3 h at 37° C. in a 5% CO₂ atmosphere. They were then rinsed with PBS and were blotted dry on cotton gauze. Moisture remaining on the insides of the inserts was removed by use of cotton buds, after which each insert and tissue was immersed in 6 mL of propan-2-ol and soaked overnight in the dark in a sealed 20 mL scintillation vial. The resultant purple solutions of formazan dye were transferred to disposable cuvettes and their absorbances at 570 nm and 650 nm (reference wavelength) were measured in an Ultrospec UV-visible spectrophotometer (LKB). Data analysis involved subtraction of the absorbance at the reference wavelength from the 570 nm reading to give corrected absorbance values. These were converted to % viability values by dividing each absorbance value by the mean of the negative control group (PBS without exposure to 0.5% SDS) and multiplying by 100.

### Histology (Efficacy)

Tissues were fixed in neutral-buffered formalin (LabServ) and were sent to Gribbles Veterinary Lab (Christchurch, NZ) for preparation of H&E-stained sections. Light microscopy was performed on a Leica DM6000 B microscope (Leica Microsystems, Switzerland) and images were acquired using Leica Application Suite v4.12 software.

### Cytokine Quantification for Anti-Inflammatory Effect (Efficacy)

For quantification of IL-1β, IFN-α2, IFN-γ, TNF-α, MCP-1 (CCL2), IL-6, IL-8 (CXCL8), IL-10, IL-12p70, IL-17A, IL-18, IL-23, and IL-33, bead-based multiplex LEGENDplex™ analysis (LEGENDplex™ Human Inflammation Panel 1 (13-plex); BioLegend, San Diego, CA, USA) was used according to the manufacturer's instructions. The relevance of each of the measured cytokines for which activity was observed is described in Table 6. Reactions were performed in duplicate. Analysis was performed with the Cytek™ Aurora flow cytometer (Cytek Biosciences Inc., Fremont, CA, USA). Data were analysed via Legendplex V8.0 software (BioLegend) and specified as pg/mL.

TABLE 6
Detailed description of immune markers measured in the study

| | |
|---|---|
| IL-6 | Interleukin-6 (IL-6) acts as both a pro-inflammatory cytokine and an anti- |
| | inflammatory myokine. IL-6 is secreted by T cells and macrophages to |
| | stimulate immune responses. It is known to be involved in wound healing. |
| IL-8 | Interleukin-8 (IL-8) is a potent chemotactic and proinflammatory cytokine, |
| (CXCL8) | produced in the skin by a variety of cells in response to inflammatory |
| | stimuli. IL-8 promotes dendritic cells migration and recruitment of |
| | monocytes and neutrophils as key steps in the initiation phase of cutaneous |
| | inflammation (Coquette et al., 2003). |
| IL-18 | Interleukin-18 (IL-18) is a pro-inflammatory cytokine, also known as |
| | interferon-γ-inducing factor. Produced by keratinocytes, where it involved |
| | in the induction of inflammatory mediators (TNF-α) as well as regulation of |
| | the cytotoxic activity of Natural Killer (NK) cells and T cells. Furthermore, |
| | IL-18 was found to support the differentiation and activation of different T |
| | helper (Th) cell subsets depending on the surrounding cytokine profile. |
| | Reviewed by Wittmann et al. (2009). Important in autoimmune |
| | inflammatory diseases e.g., lupus. Reviewed by Sedimbi et al. (2013). |

### Statistical Analysis

Statistical analysis and graphing were performed using Prism 9.2.0 (GraphPad Software). Data were analysed by one-way or two-way analysis of variance (ANOVA), with correction for multiple comparisons using Dunnett's or Šidák's method, as appropriate.

### Tolerability

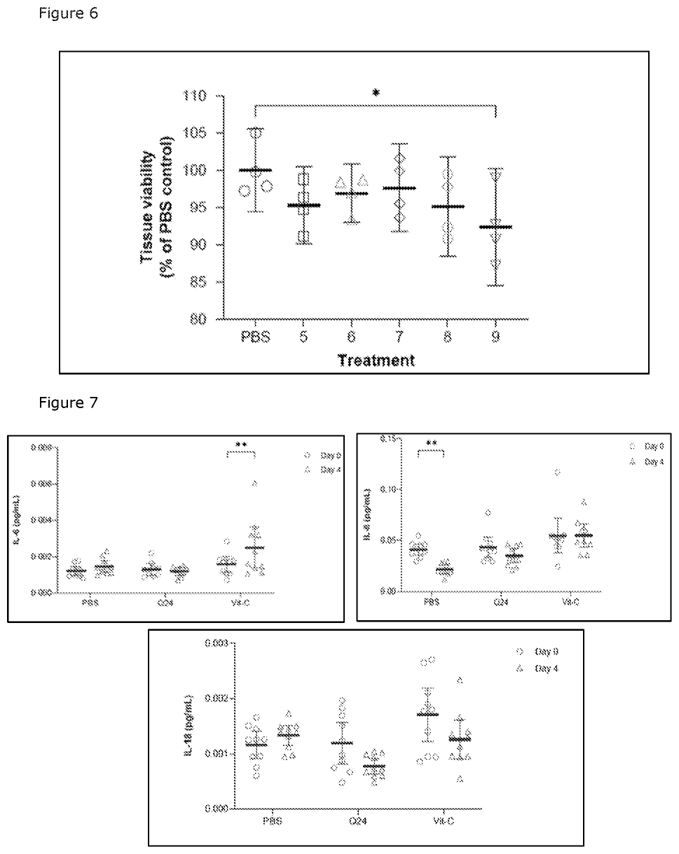

Tolerability of Micrococcus luteus Q24 was determined via dose response experiment. EpiDerm tissues treated with live Micrococcus luteus Q24 (1.42×10⁵, 1.31×10⁶, 1.46×10⁷, 1.71×10⁸ and 1.63×10⁹ cfu/ml) suspension in phosphate-buffered saline (PBS) showed >90% viability (FIG. 6). Although viability for 1×10⁹ CFU/ml was significantly different from PBS control, the mean % cells were still >90% viable indicating that Micrococcus luteus Q24 was well tolerated by the model skin tissues during the 24 h exposure.

These in vitro results compare well with the Micrococcus luteus Q24 Hydration serum in vivo trials where no skin irritation was observed even after repeated dosing with 1.5×10⁸ CFU/dose.

Conclusion: Micrococcus luteus Q24 in different clinically relevant doses is well tolerated in tissue culture model indicating safety of the strain and the products containing Micrococcus luteus Q24.

### Safety

Following the dose response experiment a dose of 1×10⁸ CFU/ml was selected for safety and efficacy studies.

Measurement of 13 cytokines (day 0 and 4) was carried out in EpiDerm tissues treated with Micrococcus luteus Q24 (1×10⁸ CFU/ml), PBS (negative control and Vit C (positive control). Out of 13 treatment related effect was observed only 3 cytokines. The interleukin-6 (IL-6), interleukin-8 (IL-8) and interleukin-18 (IL-18) are pro inflammatory cytokines known to be produced by the skin cells in response to the inflammatory stimuli. IL-6 levels showed a slight but significant increase on day 4 for vitamin C but were unchanged between the two days for Micrococcus luteus Q24 and PBS (FIG. 7 top left). IL-8 levels were slightly lower on day 4 for Micrococcus luteus Q24 and PBS (although the change was only significant for the latter), and unchanged for vitamin C (FIG. 7 top right). For IL-18, there were no significant differences in levels on days 0 and 4, although Micrococcus luteus Q24 and vitamin C showed a trend for lower values on day 4 (FIG. 7 bottom middle).

Conclusion: The results indicate that Micrococcus luteus Q24 is safe as it did not elicit in an inflammatory immune response in Epiderm tissues following daily administration for 4 days.

### Efficacy (Histology)

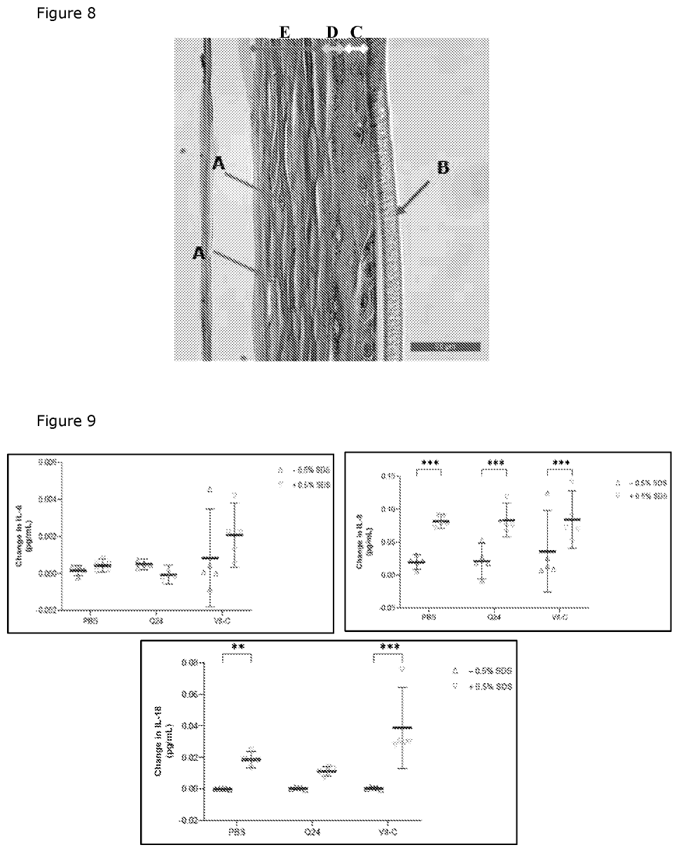

A dose of 1×10⁸ CFU/ml of live Micrococcus luteus Q24 suspension in PBS, PBS (negative control) and Vit C (positive control) was applied to the EpiDerm cells (duration 4 days) and treatment related changes to tissue morphology were recorded (n=1, FIG. 8). Although, no obvious treatment-related differences were apparent, the tissue that received Micrococcus luteus Q24 revealed nuclear remnants caught in the stratum corneum which indicates possible acceleration of stratum corneum production. The stratum corneum was also slightly thicker in the Micrococcus luteus Q24 tissue than in those treated with PBS or vitamin C. More replicates are required to confirm these findings but there seem to be a positive effect of Micrococcus luteus Q24. It is known that the stratum corneum primarily functions as a barrier between the deeper layers of skin and the outside environment, preventing toxins and bacteria from entering the body. It also helps to keep moisture from evaporating into the atmosphere and so is important for keeping the skin hydrated.

This result indicates that Micrococcus luteus Q24 may help in rejuvenating and hydrating the skin by accelerating the formation of stratum corneum.

Conclusion: Micrococcus luteus Q24 is effective in accelerating the growth of stratum corneum responsible for barrier function and moisture retention. These in vitro results substantiate the findings of in vivo trials where increase in hydration and reduction in pores and wrinkles observed by the study participants upon application of Micrococcus luteus Q24 hydration serum.

### Anti-Inflammatory Effect

Only IL-6, IL-8 and IL-18 had detectable levels to determine the effect of treatment Micrococcus luteus Q24, PBS (negative control and Vit C positive control) (FIG. 9—top left).

Pre-treatment with Micrococcus luteus Q24 caused a slight nonsignificant reduction in the amount of IL-6 released in response to 0.5% SDS, in contrast, the vitamin C-treated tissues displayed a slight increase on day 5 in response to the 0.5% SDS exposure. Significant increases in IL-8 levels were evident on day 5 compared to day 4 in response to 0.5% SDS for all three main treatment groups (FIG. 9—top right). IL-18 release was also stimulated by 0.5% SDS in all groups, but the change was not significant for the Micrococcus luteus Q24 group (FIG. 9—bottom middle).

Conclusion: The slight reduction in the SDS-induced release of IL-6 and the blunted increase in IL-18 for the Micrococcus luteus Q24+0.5% SDS group, indicates the anti-inflammatory effect of Micrococcus luteus Q24.

### Example 9: Sensory Trial

The sensory properties of balm formulations based on different platforms (beeswax, beeswax and cetostearyl alcohol, self-emulsifying glyceryl monostearate, and candelilla wax) was evaluated in a sensory trial with healthy human volunteers. In this single blind study, four lead formulations BLT-BB9, BLT-BB15, BLT-BB14 and BLT-BB18 were randomly assigned with a letter A, B, C or D, for anonymity and handed over to the participants. The participants were asked to evaluate sensory and physical properties of the formulations by means of a questionnaire by applying formulations on the back of the palm and score (1 to 5, where 1=very bad, 5=very good) for the following parameters: ease of application, viscosity, spreadability, grittiness, waxiness, greasiness, adsorption, hydration, skin feeling post application, smell, and appearance. The rankings of these properties for the four different balms were then compared.

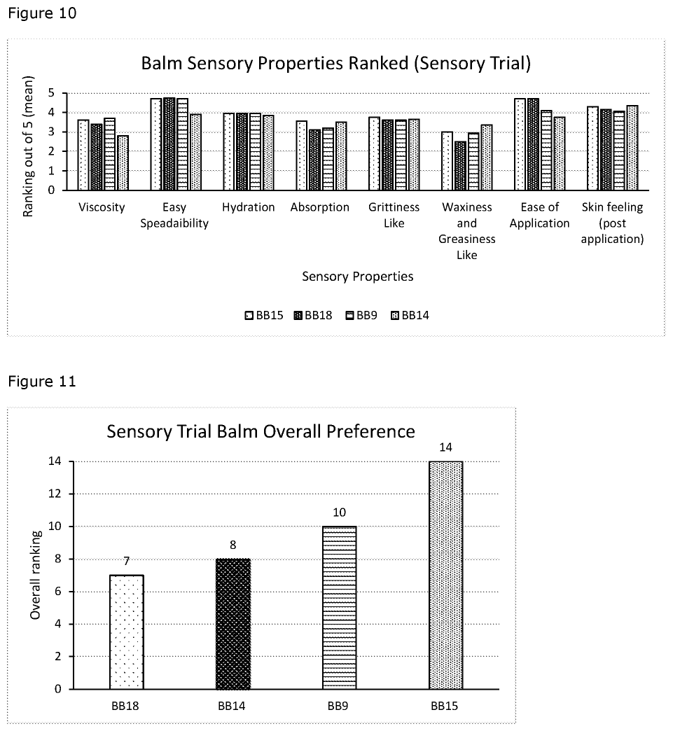

During the sensory trial, four different balm formulations were ranked by participants on various properties. As shown in FIG. 10, all four balm formulations (BB9, BB14, BB15 and BB18) were ranked 3 or more, suggesting that all formulations were well accepted by the participants. This trend was consistent for the other properties recorded (appearance, and smell) whereby similar rankings were observed.

To assist in distinguishing the balms, the respective sensory properties were ranked and for a given balm formulation the total number of properties identified as ‘highest-ranking’ was tallied, as shown in FIG. 11. For example, for ‘ease of application’ BLT-BB15 and BLT-BB18 had an average ranking of 4.7 out of 5, whereas BLT-BB9 and BLT-BB14 had an average ranking of 4.1 and 3.75 respectively out of 5. As a result, BLT-BB15 and BLT-BB18 were identified as preferred formulations for this property.

Formulation BLT-BB15 ranked highest in overall sensory performance (score of 14). While BLT-BB9 ranked highly (score of 10, FIG. 11), it was not continued for further evaluation, with the higher-ranking BLT-BB15 having the same beeswax platform instead being chosen. Plant-based formulation BLT-BB14 was also selected for further evaluation in the sensory and efficacy trial.

### Example 10: Sensory and Efficacy Trial

A single-blind randomized controlled in-house study was carried out with 12 healthy humans. The balms used in this trial have a 1% concentration of Micrococcus luteus Q24 ingredient (approximately 1E+9 cfu/g per dose). Participants were provided either Balm A (BLT-BB14) or Balm B (BLT-BB15) to be applied twice daily (morning and night) over a 7-day period on the left forearm (main area) and another area of concern (Table 7). This was selected by the individual as a location of dryness/unhealthy skin (left elbow, left face-cheek, left leg, or left upper arm).

TABLE 7
Participant information (Sensory and Efficacy Trial). Age Group
A ranges from ages 21 to 32. Age Group B ranges from 38 to 50.

| Participant | | | | | | Balm A | |
|---|---|---|---|---|---|---|---|
| Number | Sex | Age | Group | Main Area | Area of Concern | or B | Formulation |
| 1 | M | 45 | B | Forearm | Left elbow | A | BLT-BB14 |
| 2 | M | 21 | A | Forearm | Left elbow | A | BLT-BB14 |
| 3 | F | 50 | B | Forearm | Left outer leg | A | BLT-BB14 |
| 4 | F | 40 | B | Forearm | Left elbow | A | BLT-BB14 |
| 5 | F | 29 | A | Forearm | Left elbow | A | BLT-BB14 |
| 6 | F | 21 | A | Forearm | Left hand eczema | A | BLT-BB14 |
| 7 | M | 42 | B | Forearm | Left face cheek | B | BLT-BB15 |
| 8 | F | 50 | B | Forearm | Left elbow | B | BLT-BB15 |
| 9 | F | 38 | B | Forearm | Left upper arm | B | BLT-BB15 |
| 10 | F | 32 | A | Forearm | Left elbow | B | BLT-BB15 |
| 11 | F | 21 | A | Forearm | Left elbow | B | BLT-BB15 |
| 12 | F | 21 | A | Forearm | Left elbow | B | BLT-BB15 |

Skin parameters (moisture, keratin, sebum, spots, wrinkles, pores, and impurities) were measured at Day 0, 4 and 7 of the trial. These were recorded using an advanced non-invasive Skin Analyzer device (dpViso, Chowis, South Korea).

When testing, the same area was measured each time. However, this was more challenging with an awkward space such as the elbow. To minimize variation, participants placed their left hand on their left shoulder, and raised their elbow up to a 90-degree angle. In addition to the skin analyzer, keratin strips were used to test the keratin parameter. Strips were placed sticky side down on the area of concern with light pressure applied. This was left for 10 seconds before removing to analyze using the skin analyzer (DermoPrime Viso, CHOWIS, South Korea) and DermoPrime software.

Comparison of the data recorded can be used to indicate improvement in skin parameters at a specific site compared to baseline and between different balm groups. In addition, a survey was completed by the participants post-study to evaluate sensory properties of the formulations.

The statistical analysis was performed using MS Excel. Student's t-test for Paired Two Sample for Means was used to compare the participants data for different days and different formulations and the level of significance was taken as p≤0.05). During statistical analysis the level of significance was denoted by:

$$ns = not significant$$

$$p \leq 0. 0 {5}^{*} = a p - value less than 0.05$$

$$p \leq 0. 0 {5}^{* *} = a p - value less than 0.01$$

$$p \leq 0. 0 {5}^{* * *} = a p - value less than 0.001$$

$$p \leq 0. 0 {5}^{* * * *} = a p - value less than 0.0001$$

As well as investigating changes in skin parameters due to the balm formulation, as the trial investigated sensory properties of the balms, specifically what the participants thought about using the balms for an extended amount of time. There was no statistically significant difference observed between the ages of individuals applying Balm A, or Balm B (p≤0.05).

There was a total of 12 participants in the study, referred to by their participant number (P1-P12), who have varying characteristics (age and gender) (Table 7). The 12 participants were split into two groups, and assigned to use either Balm A or Balm B (Table 7). The mean ages of these two groups are 34.3 and 34 years respectively. Using a One-Way Factor ANOVA, the ages of individuals applying Balm A and B was compared and a p-value of 0.9 was produced, confirming that there is no statistical difference between the ages of these two groups.

Data of participant 6 (P6) was identified as an outlier and omitted from analysis. The same approach was taken with data outliers relating to impurities. For the other skin parameters, data outliers were not removed as these were less common, and did not tend to show a large difference to the other raw data values.

### Trial Results—Main Area (Left Forearm)

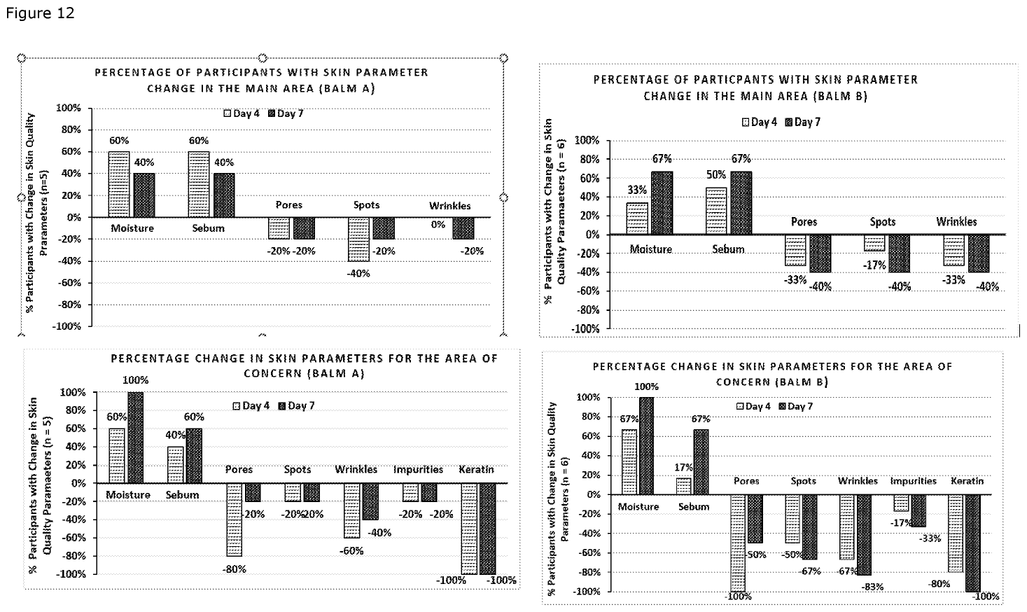

The percentage of participants displaying a change in skin parameters of the main area measured is shown in FIG. 12 for Balm A and B respectively. The data is shown in two separate bars “Day 4” and “Day 7”. Day 4 describes a change observed between Day 0 and Day 4 of the study, whereas Day 7 describes a change observed between Day 0 and Day 7.

The results for Balm A show an improvement in more participants for the skin parameters moisture, sebum and spots during the first four days of product application, whereas the results for Balm B show an improvement for more participants in all skin parameters across the 7-day trial total.

These results show that Balm A shows a skin parameter improvement more rapidly. However, the number of participants with an improvement tended to decrease by Day 7 of the trial. For example, 60% of participants showed an increase in moisture by Day 4 of the trial, however this drops to 40% of participants by Day 7 (FIG. 12).

On the other hand, Balm B shows a skin parameter improvement in more individuals between Day 0 and Day 7. For example, by Day 4 33% of participants showed an improvement in moisture, which increased to 67% by Day 7. This trend suggests that if Balm B were to be applied for longer, it may create a skin parameter improvement in more participants within the trial. Furthermore, the skin improvement is not lost by Day 7 which also suggests that Balm B is able to maintain that improvement for a longer period of time. Skin parameters Pores, Spots, Wrinkles, and Impurities for both Balm A and Balm B showed very minimal improvement over the trial period with low amounts of participants showing an improvement. This result is expected given the duration of the trial, as the forearm is a generally healthy part of skin free of defects in pores or wrinkles.

The data collected for each of the properties for the Main Area was compared to investigate whether there is a statistically significant difference between skin parameter values over time. As shown in FIG. 12, there was improvement observed for Sebum, Pores, Spots and Wrinkles, with the most notable improvement occurring within the Moisture skin parameter. For Balm B there was a statistically significant change observed between Day 0 and 7 of the trial. Interestingly, the majority of the statistically significant differences observed were in participants applying Balm B, with Balm A showing less statistically significant changes between time points. The most common trend observed is that the middle data point (recorded on Day 4), was lower or higher than that measured on Day 0 and Day 7. Therefore, over the course of the trial, for the main area most skin parameters did not change. This result is expected. The forearm is already a healthy section of the skin, with high moisture and low keratin naturally for most individuals.

### Trial Results—Area of Concern (Participant Dependent)

The area of concern was selected by the individual themselves as a place with dry or unhealthy skin, this included the left elbow, left face-cheek, left leg, or left upper arm. In addition to testing skin parameters, moisture, sebum, pores, spots, wrinkles, and impurities; keratin was also measured for this area.

As shown in FIG. 12, 100% of participants (both Balm A and Balm B) showed a decrease in the amount of keratin (dry skin) in the area of concern by Day 7 of the trial. A similar trend was observed for moisture whereby 100% of participants (both Balm A and Balm B) showed an increase in Moisture by day 7.

In addition to this, there is an increase in sebum observed for both Balm A and B. While sebum is associated with acne, it is also generally observed in a directly proportional relationship with moisture. As the moisture increases within this system, so does the sebum.

While there are individuals within the study which demonstrated an improvement in skin parameters Pores, Spots, Wrinkles, and Impurities, for both balms, Balm B showed a more notable improvement across more participants. Balm B showed more than 50% of participants with an improvement for each of these skin parameters (excluding impurities), while less than 50% of participants using Balm A experienced improvement in these skin parameters.

Overall, it can be concluded that whilst Balm B is showing a skin improvement in more individuals in Pores, Spots, Wrinkles and Impurities skin parameters, both Balm A and Balm B are showing a clear improvement in hydration (moisture) and keratin.

### Statistical Analysis—Area of Concern (Moisture and Keratin)

While the trends observed by the percentage of participants demonstrated a change is occurring, the raw data needs to be compared to better predict whether the balm is having a statistically significant improvement on skin quality parameters.

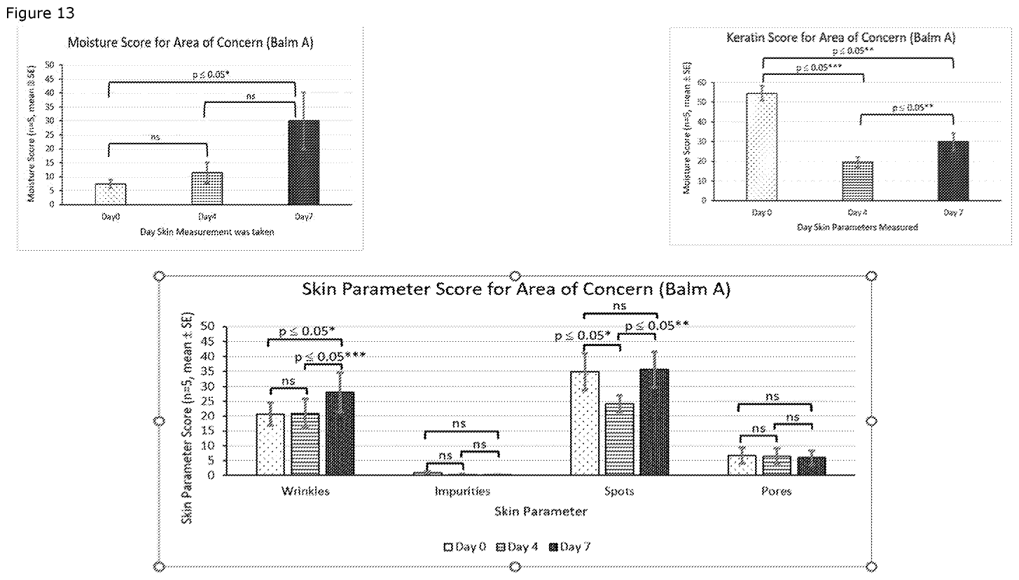

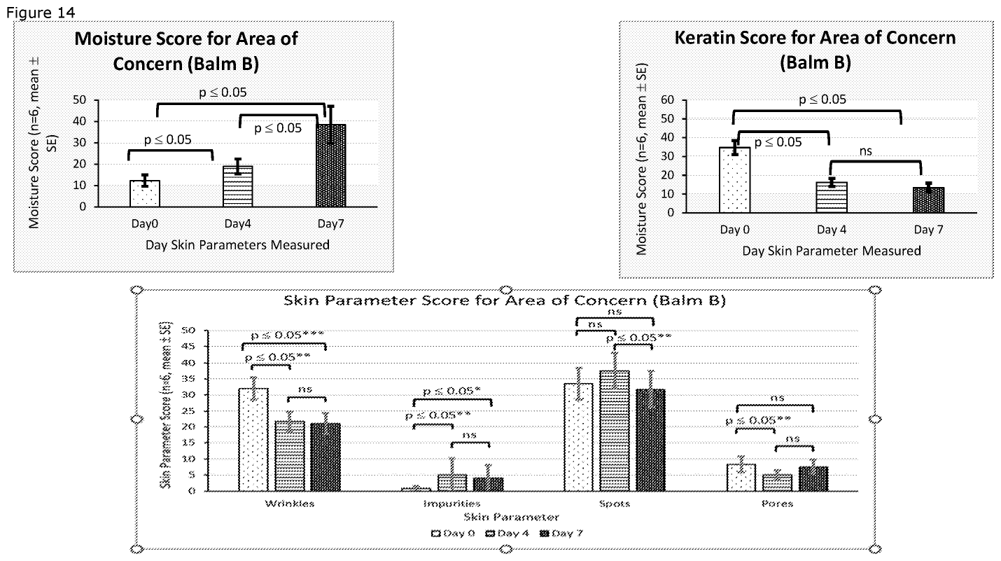

The analysis for the moisture skin parameter is shown in FIGS. 13 and 14. The moisture score for individuals using Balm A showed no statistically significant difference between Day 0 to Day 4 (P=0.2) and a weak significant difference from Day 4 to Day 7 (p=0.073). However, there is a statistically significant difference between the moisture scores for Day 0 and Day 7 (p=0.043). In other words, a significant increase in moisture in participants using Balm A was mainly observed after use of the balm for 7 days. This shows that Balm A has an advantageous hydration effect. In addition, as the moisture score is increasing over time, it can be predicted that extended use of the product will further improve the moisture skin parameter.

In contrast to the results for Balm A, the moisture score for the area of concern in participants using Balm B showed a statistically significant difference by day 4. This indicates that Balm B provided an immediate moisture improvement which was maintained throughout the 7-day trial. Similarly, Balm B moisture scores were further improved by day 7. Therefore, it can be predicted that continued use of the product would further improve the moisture skin parameter.

The consistent and statistically significant increase in moisture over time experienced by individuals using Balm B suggest that it is more successful at improving moisture over time than Balm A. This is supported by the mean moisture score being higher on Day 7 for participants using Balm B (30 and 38.5 for Balm A and Balm B respectively).

The change in score for the keratin skin parameter for Balms A and B is shown in FIGS. 13 and 14. As illustrated, there are statistically significant differences between each of the time points. for Balm A there is a decrease between Day 0 to Day 4, and a slight increase between Day 4 to Day 7, indicating that Balm A showed a decrease in dry skin immediately after use, however after a week of regular application the keratin increased slightly again. It is important to note that both Day 4 and Day 7 keratin levels are lower than Day 0 therefore an improvement is still occurring by the end of the trial.

The keratin score for Balm B showed a large decrease at the beginning (Day 0 to Day 4, p=≤0.05) similar to that observed for Balm A. The lower keratin score was maintained until Day 7.

### Statistical Analysis—Area of Concern (Other Skin Parameters)

In addition to Moisture and Keratin, other skin parameters were also investigated (Wrinkles, Impurities, Spots and Pores). These are graphed in FIGS. 13 and 14 for Balm A and Balm B respectively.

There are various statistically significant differences occurring between the skin parameters Wrinkles, Impurities, Spots and Pores for both Balm B and Balm A at Area of Concern. However, these statistically significant relationships are variable and do not seem to be showing strong trends of improvement over time. Interestingly, Balm B demonstrated a statistically significant decrease in Wrinkles.

In addition to comparing Balm A to Balm B, on the Main Area (Forearm) and Area of Concern; the results for moisture and keratin score were also analyzed by age of participant. The participants involved in the study were sorted by age and split into younger Group A (aged 21-32) and older Group B (aged 38-50) to provide two groups of similar size (Table 8).

There is a statistically significant age difference between these two groups having a mean age of 24.8 and 44.2 respectively and a p-value of 5.47E-6.

TABLE 8
Sensory and Efficacy Trial Participant
Ages (Age Group A and B Comparison)

| Participant | | | |
|---|---|---|---|
| Number | DOB | Age | Age Group |
| 3 | 1971 | 50 | B |
| 8 | 1971 | 50 | B |
| 1 | 1976 | 45 | B |
| 7 | 1979 | 42 | B |
| 4 | 1981 | 40 | B |
| 9 | 1983 | 38 | B |
| 10 | 1989 | 32 | A |
| 5 | 1992 | 29 | A |
| 2 | 2000 | 21 | A |
| 6* | 2000 | 21 | A |
| 11 | 2000 | 21 | A |
| 12 | 2000 | 21 | A |

*excluded from analysis

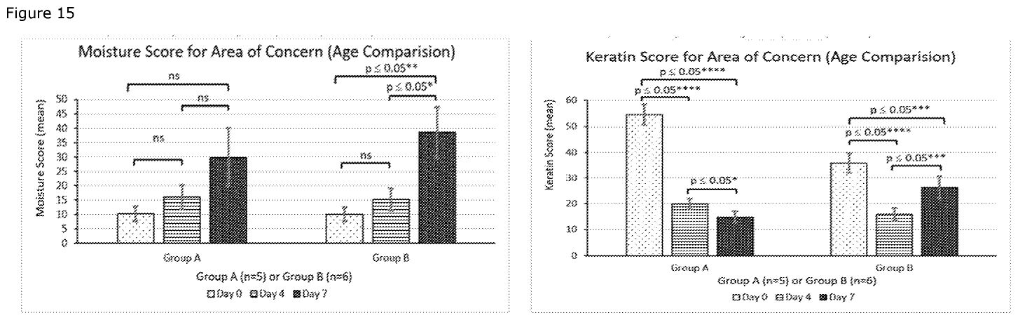

Skin parameter data for moisture and keratin for these two groups were compared to see if younger (Group A) or older (Group B) showed a statistically significant difference over the 7-day trial period. The mean data and p-values for these comparisons are described in FIG. 15.

As shown in FIG. 15 both Group A (younger) and Group B (older) show an increase in moisture score over time. Younger Group A shows a mean moisture score of 10.2, 16.0 and 29.8 over Day 0, Day 4 and Day 7 respectively, while older Group B shows a mean moisture score of 10.0, 15.2 and 38.7 over Day 0, Day 4 and Day 7 respectively. While both Groups demonstrate an increase in moisture, Group B moisture scores showed a much more statistically significant difference over time, with significant p-values (p=0.005 and p=0.014) for Day 0 to Day 7 and Day 4 to Day 7. Group A had no statistically significant difference between the three time points for moisture score. Thus, the older participants (Group B) showed a more significant increase in moisture over the trial period.

While Group A shows a gradual decrease in keratin score over time (mean score of 54.6, 19.9, and 14.9 across Day 0. Day 4 and Day 7 respectively), Group B shows a variable trend (FIG. 15). This trend demonstrates a decrease in keratin between Day 0 and Day 4 (mean keratin scores of 35.9 and 15.9 respectively), however this increases to 26.3 by Day 7. It is important to note that while there is a slight increase observed between Day 4 and Day 7, the Day 7 keratin value is still lower than the basal keratin measurement. Therefore, Keratin is still observed decreasing over time during balm application.

Both Group A and Group B showed a statistically significant difference between all-time points for keratin. This means that for both Group A and B a significant decrease in keratin was observed throughout the trial.

### Example 11: Addition of Prebiotics

### Inhibition of Common Prebiotics

CABK12 agar plates were split into segments of 6 and lawned with a suspension of Micrococcus luteus Q24 raw ingredient. Each of the aqueous potential prebiotic substances to be screened were serially diluted in a range of concentrations from 100% to 0.3% using sterile distilled water. Oil based substances were tested at 100% only.

20 μL of each concentration, of each substance were pipetted into a spot onto one segment of the lawned CABK12 agar plate and was incubated for 24 hours at 37° C. 5% CO₂ in air.

TABLE 9
Summary of prebiotic MIC results vs Micrococcus luteus Q24 and
the prebiotic potential uses based on inhibitory concentrations
Potential Prebiotics

| | Suitable to be used in | |
|---|---|---|
| Ingredient | Q24 containing product? | Appearance |
| Allantoin | Yes | Powder |
| Aloe Vera Powder | Yes | Powder |
| Colloidal Oatmeal USP organic | Yes | Powder |
| Xylitol | Yes | Powder |
| Yeast Extract | Yes | Powder |
| Fructo-oligosaccharide (FOS)- | Yes | Powder |
| Powder | | |
| Fructo-oligosaccharide (FOS)-Liquid | Yes | Oil |
| Manuka Honey Powder | Yes | Powder |
| Niacinamide | Yes | Powder |
| Green Tea Powder | No (inhibitory at all | Powder |
| | concentrations) | |
| Flaxseed Oil | Yes | Oil |
| Olive Squalene | Yes | Oil |
| Pomegranate Seed Oil-Cold pressed | Yes | Oil |
| Vitamin E, D Alpha Tocopherol | Yes | Oil |
| 1000 IU | | |
| Retinol (Vitamin A) | Yes | Oil |
| Vitamin C (Ascorbic acid) | Yes | Powder |
| Hyaluronic acid | Yes | Powder |
| Salicylic acid (SA) | No (inhibitory at >5%) | Powder |
| Lactic acid (LA) | No (inhibitory at >2.5%) | Powder |
| Calendula oil | Yes | Oil |
| Almond oil | Yes | Oil |
| Tomato oil | Yes | Oil |
| Sunscreen SPF (Neutrogena Ultra | Yes | Powder (in aqueous |
| sheer face and body) | | cream) |

Conclusion: All substances passed the first screening test for a non-Micrococcus luteus Q24 containing product except green tea powder which was inhibitory at all concentrations tested. 6 other potential prebiotics showed some inhibitory effect on Micrococcus luteus Q24 indicating they would not be suitable for use in a product requiring Live Micrococcus luteus Q24 stability, but maybe of use in a prebiotic only formulation.

Effect of Prebiotics on the Growth of Micrococcus luteus Q24

A batch of M17 broth (Difco #218561) was made, excluding the lactose solution, by following the manufacturer's instructions. 50 ml of broth was dispensed into sterile 100 ml Schott bottles and 2.5 g (5% w/v) of each potential prebiotic substance was added and mixed well using a magnetic stirrer and stir plate. The mixtures were autoclaved at 110° C. for 10 minutes and allowed to cool.

The suspensions of the prebiotic candidates were prewarmed to 40° C. in order to allow for increased homogenisation of any oil-based components prior to dispensing into the wells. 2 ml of each suspension and an M17 only (control) were pipetted into a sterile 24 well tissue culture plate. A suspension of Micrococcus luteus Q24 raw ingredient was made in PBS and adjusted to an optical density of 0.125. 100 μl of the suspension was pipetted into each well. Once the suspension was added each well was mixed by aspirating and dispensing the solution 5 times with a 1 ml pipette.

Each sample was enumerated at time points 0, 3, 6, 15, 26 and 34 hours using the following method. At each time point the wells were mixed by using a 1 ml pipette and dispensing and aspirating 5 times. Then 100 μl was removed from each well and added to a 900 μl Eppendorf of PBS (1/10 dilution). These 1/10 dilutions were vortexed horizontally for 10 minutes at 2600 rpm. Then the samples were serially diluted by transferring 100 μl of the 1/10 dilution into a fresh 900 μl PBS, this was repeated until serially diluted 6 times. 20 μl of each dilution from each sample was spotted onto a sheep blood agar plate in triplicate. The spots were left for 30 minutes to dry and placed into a 37° C., 5% CO₂ incubator for 28-36 hours. The number of colonies in each spot were then counted using an electronic colony counter and average to give the final result.

Results: All the prebiotic candidates tested in showed an increase in Micrococcus luteus Q24 higher than that of the control with most resulting in a 1 to 2-log increase over 24 hours compared to the control.

Conclusion: Surprisingly commonly used prebiotics (carbohydrates (except for Oatmeal)) typical for topical application have not supported growth of Micrococcus luteus Q24.

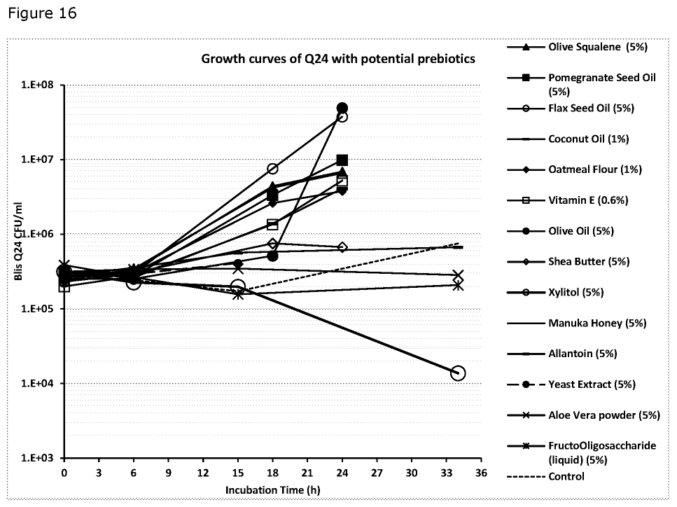

Conventional substances used as prebiotic for gut bacteria did not perform well at increasing the growth of Micrococcus luteus Q24, some such as xylitol and Manuka honey were detrimental to the live Micrococcus luteus Q24 cell count at 24 hours (FIG. 16).

In contrast oil-based substances typically used as functional actives and other cosmetic ingredients performed very well, with the best increasing the live cell count of Micrococcus luteus Q24 by 2-log over 24 hours.

### Combinations of Prebiotics

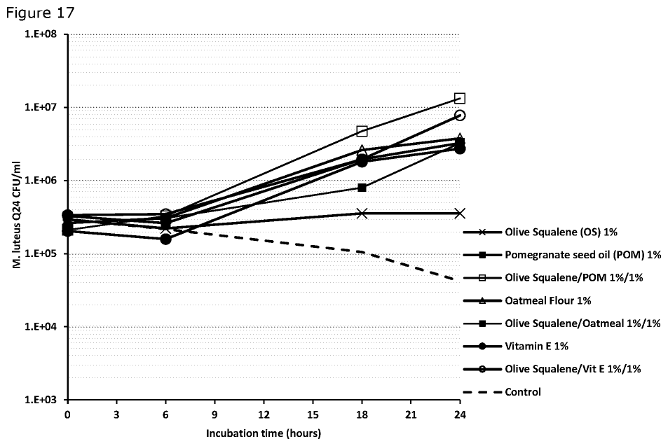

The same method was used as described above except the time points tested were 0, 6, 18 and 24 hours. Olive squalene was selected as one of the preferred prebiotics with 3 others that were analysed in combination with olive squalene and compared to the results of the prebiotic candidates alone (FIG. 17).

Results: The growth of the Micrococcus luteus Q24 was increased well above the sum of olive squalene and pomegranate seed oil separately, showing a synergistic response when both prebiotics are together resulting in an increased growth rate of Micrococcus luteus Q24.

The growth of the Micrococcus luteus Q24 was the same as the sum of olive squalene and oatmeal flour (colloidal oatmeal) separately, showing no synergistic response for this combination.

The growth of the Micrococcus luteus Q24 was increased well above the sum of olive squalene and vitamin E separately, showing a synergistic response when both prebiotics are together resulting in an increased growth rate of Micrococcus luteus Q24.

Conclusion: Both olive squalene+pomegranate seed oil and olive squalene+vitamin E were found to have a synergistic relationship increasing the growth rate of Micrococcus luteus Q24 compared to either alone. Olive squalene and oatmeal flour did not show this same response. A formulation made to include both olive squalene and either pomegranate seed oil or vitamin E will be more efficacious than a formulation including only olive squalene, pomegranate seed oil, or vitamin E.

1. A topical composition comprising Micrococcus luteus Q24 and a viscosity modifier, the composition having a viscosity of from about 2,500,000 to about 8,000,000 cP at 25° C.

2. A topical composition according to claim 1, wherein the composition comprises Micrococcus luteus Q24 in an amount of about 1×10³ to about 1×10¹² cfu/g.

3. A topical composition according to claim 1, wherein the composition comprises one or more viscosity modifiers in a combined amount of about 30 to about 80% w/w, wherein the viscosity modifier is selected from the group consisting of hydrophobic silica, hydrophilic silica, white beeswax, white beeswax with cetostearyl alcohol, yellow beeswax, yellow beeswax with cetostearyl alcohol, ethyl cellulose, emulsifying wax, cocoa butter, shea butter, fatty alcohols, glyceryl monostearate, candelilla wax, lanolin, and a combination of any two or more thereof.

4-5. (canceled)

6. A topical composition according to claim 1, wherein the composition comprises a dispersing agent.

7. A topical composition according to claim 6, wherein the composition comprises the dispersing agent in an amount of about 0.1 to about 5% w/w, wherein the dispersing agent is selected from the group consisting of polysorbate 80, polysorbate 20, sorbitan oleate, egg lecithin, soybean lecithin, polyoxyl 35 castor oil, sodium stearoyl glutamate, sodium cocoyl isethionate, cetearyl olivate, and a combination of any two or more thereof.

8. (canceled)

9. A topical composition according to claim 1, wherein the composition further comprises an oil vehicle, wherein the oil vehicle is selected from the group consisting of a medium chain triglyceride (MCT) oil, a caprylic/capric triglyceride oil, plant oil, sunflower oil, canola oil, soybean oil, olive oil, jojoba oil, argan oil, rosehip oil, marula oil, chamomile oil, tamanu oil, grapeseed oil, calendula oil, pomegranate oil, macadamia oil, buriti fruit oil, sweet almond oil, evening primrose oil, or a combination thereof.

10-13. (canceled)

14. A topical composition according to claim 1 comprising
about 0.5 to 2% w/w Micrococcus luteus Q24 or about 1×10³ to about 1×10¹² cfu/g Micrococcus luteus Q24,
about 10 to about 35% w/w beeswax,
about 2 to about 10% w/w cetostearyl alcohol or about 10% to about 30% w/w shea butter,
about 10% to about 30% w/w cocoa butter,
a quantity sufficient amount of oil vehicle,
wherein the composition has a viscosity of from about 2,500,000 to about 8,000,000 cP at 25° C.

15-17. (canceled)

18. A topical composition according to claim 7, further comprising about 0.5 to about 2% w/w polysorbate 80.

19. A topical composition according to claim 7, wherein the composition further comprises an oil vehicle, and wherein the oil vehicle is a medium chain triglyceride oil, or olive oil.

20. A topical composition according to claim 1, further comprising 0.1 to 35% w/w prebiotic(s), wherein the prebiotic(s) is selected from olive squalene, pomegranate seed oil, vitamin E, or a combination of olive squalene and pomegranate seed oil; pomegranate seed oil and oatmeal; olive squalene and vitamin E; or olive squalene and pomegranate seed oil and vitamin E.

21. A topical composition according to claim 20 wherein the prebiotic(s) is selected from olive squalene, pomegranate seed oil, vitamin E, or a combination thereof.

22. A topical composition according to claim 20 wherein the prebiotic(s) is selected from a combination of olive squalene and pomegranate seed oil; pomegranate seed oil and oatmeal; olive squalene and vitamin E; and olive squalene and pomegranate seed oil and vitamin E.

23. A topical composition according to claim 1, wherein Micrococcus luteus Q24 is lyoprotectant-free and/or is preservative-free.

24. (canceled)

25. A topical composition according to claim 1, wherein the composition further comprises one or more additional probiotic(s), wherein the one or more additional probiotic(s) is selected from the group consisting of a Streptococcus spp., a Lactobacillus spp., Limosilactobacillus spp., a Lacticaseibacillus spp., a Ligilactobacillus spp., Lactiplantibacillus spp., a Bifidobacterium spp., a Saccharomyces spp., and a combination of any two or more thereof, and wherein the Streptococcus spp. is selected from the group consisting of Streptococcus salivarius K12, Streptococcus salivarius M18, Streptococcus 24SMB, Streptococcus oralis (such as S. oralis 89a), Streptococcus salivarius DB-B5, and a combination of any two or more thereof.

26-27. (canceled)

28. A topical composition according to claim 25, wherein the composition comprises each additional probiotic in an amount of about 1×10³ to about 1×1012 cfu/g.

29. A topical composition according to claim 1, wherein the composition further comprises one or more postbiotic(s), wherein the postbiotic is selected from the group consisting of Streptococcus ferment, Streptococcus ferment filtrate, Streptococcus ferment lysate, Streptococcus lysate, Streptococcus filtrate, Lactobacillus (including Limosilactobacillus spp., Ligilactobacillus spp., and Lactiplantibacillus spp.) ferment, Lactobacillus (including Limosilactobacillus spp., Ligilactobacillus spp., and Lactiplantibacillus spp.) filtrate, Micrococcus ferment lysate, Bifidobacterium ferment lysate, Bifidobacterium ferment filtrate, Galactomyces ferment filtrate, Saccharomyces ferment filtrate, Bacillus ferment, Bacillus filtrate, and a combination of any two or more thereof.

30. (canceled)

31. A topical composition according to claim 1, wherein the composition further comprises an inhibitory activity enhancer, a buffering agent, an antibacterial agent, a prebiotic, a fragrance, an antioxidant, a colourant, a skin protective agent, an antimicrobial, an aluminium salt, a mineral pigment, an odour absorbant or neutraliser, a sunscreen agent, and a combination of any two or more thereof,
wherein the inhibitory activity enhancer is selected from the group consisting of sodium chloride, ethylenediaminetetraacetic acid, arginine, calcium carbonate, and a combination of any two or more thereof,
wherein the buffering agent is selected from the group consisting of calcium carbonate, magnesium carbonate, sodium hydrogen phosphate, sodium dihydrogen phosphate, dipotassium hydrogen phosphate, potassium dihydrogen phosphate, magnesium carbonate, urea, hydrated aluminium oxide, bentonite clay, kaolin clay, and a combination thereof,
wherein the antibacterial agent is selected from the group consisting of xylitol, erythritol, epidermin, nisin, salivaricin A, salivaricin A1, salivaricin A2, salivaricin B, salivaricin 9, salivaricin MPS, salivaricin D, salivaricin M, salivaricin G32, salivaricin A5, and a combination thereof,
wherein the prebiotic is selected from the group consisting of Manuka honey powder, olive squalene, pomegranate seed oil, flax seed oil, coconut oil, colloidal oatmeal, vitamin E, vitamin C, retinol (vitamin A), olive oil, hyaluronic acid, calendula oil, almond oil, tomato oil, allantoin, aloe vera powder, inulin, Borago officinalis (borage) seed oil, pomegranate extract, monk fruit extract, xylitol, yeast extract, fructooligosaccharide powder, fructooligosaccharide liquid, fructooligosaccharides, galactooligosaccharides, xylooligosaccharides, niacinamide, sunscreen, and a combination of any two or more thereof,
wherein the fragrance is selected from the group consisting of rose water, orange blossom, rose gardenia, peony, white jasmine, ylang ylang oil, geranium oil, rose oil, and a combination of any two or more thereof,
wherein the antioxidant is selected from the group consisting of vitamin E, resveratrol, squalene, vitamin C, β-carotene, retinyl acetate, retinyl palmitate, retinol, niacinamide, caffeine, bakuchiol, licorice root, and a combination of any two or more thereof,
wherein the skin protective agent is selected from the group consisting of ceramide, collagen, elastin, coenzyme Q10, hydrolysed collagen, hyaluronic acid, sodium hyaluronate, retinol, palmitoyl tripeptide-1, palmitoyl tetrapeptide-7, palmitoyl tetrapeptide-38, acetyl hexapeptide-8, heptapeptide-14, heptapeptide-15-palmitate, palmitoyl tetrapeptide-7, palmitoyl tripeptide-1, alpha-hydroxy acids, beta-hydroxy acids, vitamin B5, seaweed, seaweed extract, silicone, xylitol, oat extract, oatmeal powder, colloidal oatmeal, aloe vera, sunscreen, lactic acid, camellia oil, hemp seed oil, sea buckthorn fruit oil, lanolin, and a combination of any two or more thereof, and
wherein the antimicrobial is selected from the group consisting of zinc, azelaic acid, benzoyl peroxide, tea tree oil, salicylic acid, and a combination thereof.

32-39. (canceled)

40. A topical composition according to claim 1, wherein the composition further comprises from about 0.1% to about 10% w/w olive squalene, and/or from about 0.1 to about 10% w/w pomegranate seed oil, and/or from about 0.1 to about 3% w/w vitamin E.

41. (canceled)

42. A topical composition according to claim 1, wherein the composition is non-aqueous.

43. A topical composition according to claim 1, wherein the particle size (Dv90) of Micrococcus luiteus Q24 is less than about 300 μm, less than about 250 μm, or less than about 100 μm.

44. (canceled)

45. A topical composition according to claim 1, wherein the composition has a shelf-life of at least 3 months at 25° C., at 60% RH.

46. A method to improve appearance of skin or at least one sign of aging comprising applying to the skin a topical composition according to claim 1, wherein improving the appearance of skin or at least one sign of aging includes that skin looks more radiant, skin looks healthier, skin feels more hydrated, pores are reduced in size, skin feels softer, skin looks clearer, wrinkles are reduced, dryness is reduced, spots are reduced, impurities are reduced, moisture is increased, keratin is reduced, sebum production is modulated, and improving at least one of teething rash, nappy rash, or dry skin in infants.

47-51. (canceled)

52. A method of manufacturing a topical composition comprising Micrococcus luteus Q24 comprising the steps of:
a) heating a mixture of an oil vehicle and a viscosity modifier to provide a melt mixture,
b) cooling the melt mixture to a temperature in the range of about 35° C. to about 28° C., or cooling the melt mixture to a temperature between about 22° C. to about 27° C.
c) adding Micrococcus luteus Q24 to the melt mixture from step b) to provide a uniform mixture,
d) cooling the mixture from step c) to room temperature to provide the composition.

53-55. (canceled)

56. A method according to claim 52, further comprising adding a dispersing agent during step (a) or before step (b).

57. (canceled)
